(19) United States
(12) Patent Application Publication
(10) Pub. No.: US 2018/0127709 A1
(43) Pub. Date: May 10, 2018

(54) MULTISCALE HIERARCHICAL SCAFFOLD

(71) Applicant: Sharmila M. Mukhopadhyay, Beavercreek, OH (US)

(72) Inventor: Sharmila M. Mukhopadhyay, Beavercreek, OH (US)

(73) Assignee: Wright State University, Dayton, OH (US)

(21) Appl. No.: 15/862,295

(22) Filed: Jan. 4, 2018

Related U.S. Application Data
(63) Continuation-in-part of application No. 14/277,205, filed on May 14, 2014.
Application No. 14/277,205 is a continuation-in-part of application No. 13/681,752, filed on Nov. 20, 2012.
(60) Provisional application No. 62/442,236, filed on Jan. 4, 2017.
(60) Provisional application No. 61/563,152, filed on Nov. 23, 2011.

(51) Int. Cl. C12N 5/00; C23C 16/511; C23C 16/455; C23C 16/56; C23C 14/58

(52) U.S. Cl. CPC C12N 5/0068; C23C 16/511; C23C 16/45525; C12N 2535/00; C23C 14/5806; C12N 2539/00; C12N 2533/00; C23C 16/56

(57) ABSTRACT

A multiscale hierarchical scaffold with the complexity of extracellular matrix (ECM) is disclosed. The multiscale hierarchical scaffold may be a carbon-based scaffold with hierarchical nanoscale and microscale architecture with controlled physicochemical properties. More specifically, multiscale hierarchy is built by growing carbon nanotube carpets on two types of scaffolds, namely, interconnected microporous carbon foams and aligned carbon fiber mats.

### CROSS-REFERENCE TO RELATED APPLICATION

This application claims the benefit of U.S. Provisional Patent Application No. 62/442,236 entitled “MULTISCALE HIERARCHICAL SCAFFOLD FOR TISSUE REGENERATION” and filed on Jan. 4, 2017. This application is also a Continuation-in-Part of pending U.S. patent application Ser. No. 14/277,205 entitled “CONTROLLING SURFACE WETTABILITY OF ULTRAHIGH SURFACE AREA HIERARCHICAL SUPPORTS” and filed on May 14, 2014, which a is a Continuation-in-Part of pending U.S. patent application Ser. No. 13/681,752 entitled ‘ULTRAHIGH SURFACE AREA SUPPORTS FOR NANOMATERIAL ATTACHMENT’ and filed Nov. 20, 2012, which claims the benefit of U.S. Provisional Patent Application No. 61/563,152 entitled ‘ULTRAHIGH SURFACE AREA SUPPORTS FOR NANOMATERIAL ATTACHMENT’ and filed Nov. 23, 2011. The entireties of the above-noted applications are incorporated by reference herein.

### TECHNICAL FIELD

The present innovation relates to a hierarchical structure wherein a conventional compact or porous solid substrate is modified and further includes nanoscale attachments that significantly increase its surface area, as well as coatings on such nanoscale attachments to alter the properties of the nanoscale attachments, and provides advantages in surface-related applications such as catalysis, sensing, bioscaffolding, charge and gas storage, chemical, thermal and mechanical interactions. The innovation also relates to strategies for stimulating regeneration of tissue, including functional muscles. More specifically, regenerative stimulation strategies involving surface nano-structuring and functionalization are shown to cause improved differentiation in cultured stem cells. As a specific example, strategies are aimed at promoting differentiation of progenitor cells into multinucleated differentiated tissues, a key initial step in functional tissue regeneration.

### BACKGROUND

Prior patents have taught that carbon nanotubes and/or nanometer-scale metals can be grown using various methods. U.S. Pat. No. 7,148,512 discloses carbon nanotubes having nanometer-scale silver filling embedded on a silver colloid base to create a thermal interface for heat conduction in electronic components such as semiconductor chips. U.S. Pat. No. 8,207,658 discloses carbon nanotubes may be grown on metal surfaces, where the metal surface is preferably a nonmagnetic metal alloy, such as INCONEL 600. U.S. Pat. No. 7,438,885 teaches carbon nanotubes filled and decorated with palladium nanoparticles, wherein the synthesis involves arc-discharge technique with palladium chloride solution at temperatures greater than 3000° C. The palladium is ionized into nanoparticles and the graphite electrodes generate layers of graphene (carbon) which roll away from the anode and encapsulate or entrap the Pd-nanoparticles for use potentially as a gas sensor or as a means for hydrogen storage. U.S. Pat. No. 8,052,951 discloses bulk support media such as quartz fibers or particles, carbon fibers, or activated carbon, having carbon nanotubes formed therewith adjacent to metal catalyst species. Methods employ metal salt solutions such as iron chloride, aluminum chloride, or nickel chloride. The carbon nanotubes may be separated from the bulk support media and the metal catalyst species by using concentrated acids to oxidize the media and catalyst species.

Previously, nanomaterials such as nanotubes and nanoparticles that are not grown on metals or silicon wafers are either in the form of loose dust or entrapped in the pores. The resultant structures were then comprised of loosely combined units that cannot effectively restrain nanoparticles to prevent loss or leaching in service conditions.

Owing to the rise in demand for catalysis, sensing and environmental clean-up materials, there is a growing need for ultra-high surface area solids capable of more extensive interaction with the surrounding medium (air, water, biological fluids etc.). Two important parameters that influence these interactions are specific surface area (SSA) and interfacial energy. Materials with high surface area, such as high porosity solid structures and related articles discussed in connection with the subject innovation can provide for increased interaction with surrounding media for a variety of applications. One limitation is that the entire surface and related functionalities of graphitic materials may be under-utilized due to the hydrophobic nature of graphitic carbon surfaces, which may prevent effective contact with polar fluids such as water-based liquids. Graphite itself is non-polar and hydrophobic.

The subject innovation addresses these challenges by strongly anchoring nanoscale attachments onto surfaces of larger solids, thereby ensuring that the nanoscale attachments do not coalesce during transportation and storage and further ensuring they are not lost to the environment, thus preventing detrimental effects to the environment. The subject innovation synthesizes structures wherein attachment between nanoparticles and the parent substrate, including porous substrates, are at least as strong as the substrate itself. The subject innovation also provides for modification of these techniques and substrates to develop methods suitable for controlling the wettability of these hierarchical multi-scale carbon materials

Recently, carbon-based materials are gaining popularity in various tissue engineering applications such as osteogenesis, neural cell growth, bone implants and drug delivery. For example, three-dimensional macro-porous graphene foams can provide electrically conductive and macro-porous surface and can promote proliferation and differentiation of neural stem cells.

Similarly, macro-porous architecture of the scaffold may be advantageous for easy exchange of nutrients and waste products as well as facilitating cell infiltration throughout the scaffold volume. Honeycomb-like polymer foams have been investigated for cardiac muscle regeneration and interconnected honey-comb architecture was thought to reinforce the scaffold during the continuous contraction and relaxation process of myocardial tissue.

Recent tissue engineering approaches utilize various scaffolds ranging from decellularized matrices to aligned biomaterials. It is known in the art that tissue formation can occur in swirled and unorganized patterns. Such unorganized, non-aligned cellular structures, however, may hamper fusion and formation of organized tissues with the cellular alignment necessary for functionality (e.g., multinucleated myotubes) and consequently, may hamper functional regeneration. Aligned structural or topographical cues provide contact guidance, where myotubes can be formed parallel to each other leading to functional tissue development.

Although, prior attempts have focused on nano- or microscale organization, a systematic approach to build the multiscale hierarchy into scaffolds is lacking.

Surface hydrophobicity/hydrophilicity, or surface wettability (as may be measured by contact angle), is a factor that affects cell adhesion on a scaffold surface. For example, moderately water-wettable surfaces with contact angles around 70° facilitate better attachment of some cells to the scaffolds through strong binding of cellular adhesion proteins. Hydrophobic, however, tend to promote the adsorption of proteins such as laminin and fibronectin, which further disrupt ordered water molecule, leading to increased entropy. Such entropy-driven adsorption is usually strong and irreversible compared to enthalpy-driven and reversible adsorption on easily wettable surfaces. Protein adsorption further plays important role in cell adhesion in some tissues. Surface nano-functionalization with carbon nanotube is known to increase the contact angle of carbon foam surfaces.

Roughness is another factor that may affect wettability as well as mechanical cell adhesion on a scaffold surface. Increase in contact angle and decrease in surface wetting was also reported for poly-L-lactic acid surfaces coated with multiwall carbon nanotubes. Here decrease in wettability of carbon nanotube-coated poly-L-lactic acid surfaces was attributed to nano-roughness.

Papenburg et al. reports surface roughness plays a more important role on cell attachment and proliferation than surface hydrophobicity. Nano-roughness has been shown to influence cell attachment and proliferation by facilitating better cell-material and cell-cell interaction.

Electrically conductive carbon-based scaffolds have been widely used as conductive materials in energy storage-related applications.

For neurological applications, carbon based nanostructures such as multi-walled carbon nanotubes (MWCNT) have dimensions and properties that match the neurological network that can help with synapse formation, cell signaling and other favorable biophysical interactions.

Conductivity may be a factor in cell-cell interactions. Enhanced differentiation may also be due to enhanced cell-cell communication as many cells, including neuroblasts and myoblasts need electrically conductive surface for the development of functional tissue and conductive carbon nanotube nanostructures may enhance transmission of such signals as suggested by the art. Indeed, prior art studies with conductive materials have shown to improve differentiation into of muscle, nerve, cardiac, liver, and bone cells into more organized functional units.

The current innovation builds on these advances while avoiding some of currently recognized drawbacks, and incorporates in part carbon nanotubes for surface functionalization as they have been shown to promote myoblast differentiation and neural web formation. Along with electrical conductivity, carbon nanotubes also impart nano-roughness which may be beneficial for cell adhesion and spreading. Indeed, when encapsulated in gelatin methacrylate hydrogels, carbon nanotubes have promoted spreading and elongation of cardiomyocytes.

The synthesizing process of carbon nanotube carpets may use a two-step process; first step involves the coating of silica nanolayer and the second step involves growth of carbon nanotube carpets by chemical vapor deposition. This process ensures growth of immobilized carbon nanotube carpets on the surface of the scaffolds.

Although carbon nanotubes have been utilized in many biomedical applications, their cytocompatibility still remains a concern. Cytotoxic effects of carbon nanotubes have been studied extensively. Carbon nanotubes were found to be cytotoxic when they were freely suspended in culture and were available for cellular uptake. The strongly attached carbon nanotubes of this innovation are not available for cellular uptake, hence capable of overcoming this limitation

### SUMMARY

The following presents a simplified summary in order to provide a basic understanding of some aspects of the innovation. This summary is not an extensive overview of the innovation. It is not intended to identify key/critical elements or to delineate the scope of the innovation. Its sole purpose is to present some concepts of the innovation in a simplified form as a prelude to the more detailed description that is presented later.

In at least one embodiment, a hierarchical structure characterized by ultrahigh surface area comprises: a solid substrate; an intermediate layer; at least one plurality of nanoscale attachments that are strongly bonded to the intermediate layer and a functional species coating the at least one plurality of nanoscale attachments.

In one or more embodiments, the hierarchical structure further includes a substrate surface, wherein at least one aspect of the substrate surface is modified to enhance bonding.

In one or more embodiments, the intermediate layer of the hierarchical structure is formed at the substrate surface.

In one or more embodiments, the hierarchical structure is characterized by a specific surface area of at least about 100 times the specific surface area of the solid substrate.

In one or more embodiments, the solid substrate of the hierarchical structure is compact or porous.

In one or more embodiments, the porous substrate comprises fibers, foams, sponges, fabric, paper, or combinations thereof.

In one or more embodiments, the substrate is inorganic or organic.

In one or more embodiments, the substrate comprises carbon, oxides, ceramics, glasses, polymers, or combinations thereof.

In one or more embodiments, the porous substrate is derived from plant, animal, or geological sources.

In one or more embodiments, the nanoscale attachments comprise nanotubes, nanoparticles, or combinations thereof.

In one or more embodiments, the nanotubes are cylindrical structures with diameters ranging from about 10 to about 50 nm and aspect ratios ranging from about 10 to about 100,000.

In one or more embodiments, the nanotubes are useful for guiding cell growth and for efficient thermal and electrical transport with the surrounding media.

In one or more embodiments, the nanoparticles comprise metals or compounds with surface-active properties suitable for catalysis, sensing, bioactivity, or antibacterial effect.

In one or more embodiments, the nanoparticles have a controlled particle size distribution from about 3 nm to about 8 nm.

In one or more embodiments, the nanoparticles have a broad particle size distribution from about 5 nm to about 100 nm.

In one or more embodiments, the nanoparticles are elements, compounds, or combinations thereof.

In one or more embodiments, the hierarchical structure is used for water purification.

In one or more embodiments, the hierarchical structure is used for sensing and remedial treatment of gases or fluids.

In one or more embodiments, a method of fabricating a hierarchical structure comprising: selecting and preparing a parent substrate, wherein the preparing may optionally include cleaning or activation; modifying the substrate surface to form an intermediate layer; attaching at least one plurality of nanoscale attachments, wherein the nanoscale attachments are selected from nanotubes, nanoparticles, or combinations thereof, onto the intermediate layer; optionally attaching a second plurality of nanoscale attachments, wherein the nanoscale attachments are selected from nanotubes, nanoparticles, or combinations thereof, onto the first plurality of nanoscale attachments and intermediate layer; wherein the steps of forming the intermediate layer and attaching the nanoscale attachments use one or more surface modification techniques selected from the group (a) wet chemistry, (b) chemical vapor deposition, (c) plasma deposition, (d) atomic layer deposition, (e) physical vapor deposition, (f) controlled environment heating, or a combination thereof; optionally heating in a controlled environment, and coating the nanoscale attachments with functional species (e.g., for affinity to various materials, such as polar fluids (e.g., via oxygen containing species, etc.), oils, proteins, etc.).

The state of the art encounters problems with most each of the many strategies of regeneration stimulation employed. The innovation circumvents these problems by designing the regenerative strategies to recreate the microenvironment conducive for tissue formation (e.g., myogenesis, neurogenesis, and osteogenesis). Aspects of the innovation include the engineering of carbon-based foam and fiber scaffolds with multiscale hierarchy conferred by nanostructured carbon nanotube carpets and either microscale interconnected pores (Foam) or fibrils bundled into aligned fibers (Fiber). Non-functionalized foam scaffolds and carbon nanotube functionalized foam scaffolds are hereafter referred as Pristine-Foam and Carbon Nanotube-Foam, respectively (schematic in FIG. 22A). An aspect of the innovation is contact guidance. In addition to other aspects, macro- and microscale scaffold architecture provides contact guidance to myoblast cells. This may include different microscale features such as interconnected pores or aligned fibrous structure.

In one specific study involving myoblasts, the pristine-foam scaffolds showed few myosin heavy chain (hereafter “MHC”)-positive myocytes without formation of fused and continuous myotubes. On the other hand, pristine-fiber mats (as opposed to fibers alone) promoted not only differentiation but also fusion of myocytes into multinucleated myotubes.

This signifies that the innovation's scaffold architecture (interconnected porous vs aligned fibrous structure) is instrumental in facilitating fusion of myocytes to form multinucleated myotubes. In this case, controlled modulation of surface wettability by nano-functionalized carbon nanotube or silica-coated carbon nanotube revealed a minimal effect of wettability and a dominant role was played by nano-roughness in promoting differentiation into myocytes.

Nanostructured carbon nanotube carpets enhance myoblast differentiation into myocytes in both the foam and fiber scaffolds; however, it alone was not sufficient to promote fusion of myocytes into multinucleated myotubes. Indeed, nanostructured carbon nanotubes combined with aligned fibrous structure facilitated formation of multinucleated myotubes and their end-to-end fusion. This was mainly due to excellent contact guidance provided by aligned fibrous architecture along with nano-roughness and conductive surface conferred by carbon nanotube carpets.

In one aspect, the innovation engineers multiscale hierarchy into carbon-based materials with controlled nanoscale (nano-roughness, wettability) and microscale features (highly interconnected pores vs highly aligned fibers). The innovation may contain commercial carbon based substrates having suitable mechanical property (rigid foams versus flexible fibers) and microscale features (highly interconnected pores vs highly aligned fibers) and engineers multiscale hierarchy through controlled nanoscale modifications (nano-roughness, wettability and functional chemistry) of the surface. More specifically, one aspect of the innovation may process carbon materials, in an embodiment, into two types of multiscale hierarchical scaffolds; namely interconnected porous carbon foams and aligned carbon fiber mats. Example embodiments included reticulated carbon foams with 200-500 μm size micro-pores with wall width of 50-80 μm as structural repeating units while carbon microfibers (diameter 5-6 μm) were repeating units in aligned carbon fiber mats. One aspect of the innovation introduces nanoscale features by growing tailored carpet-like arrays of carbon nanotubes on the micro-pore walls of foams and surfaces of fibers. Such surface functionalization offers controlled nanoscale roughness and increased specific surface area.

Another aspect of this innovation introduces tailored physicochemical properties like wettability and protein affinity. An aspect of the innovation specifically may feature the integrated effects of multiscale hierarchy, that is, microscale features (interconnected pores vs. aligned fibers) and surface nano-functionalization (carbon nanotube/Si-carbon nanotube) as portrayed with an example of differentiation of mouse myoblasts into multinucleated myotubes. The carbon nanotube nano-functionalization of carbon-based materials may facilitate biomimetic cell-material interaction and promote differentiation of specific cells, while the highly ordered fiber alignment may enhance fusion of differentiated cells (e.g., myocytes) into multinucleated tissue (e.g., muscle tissue) or building blocks for tissue (e.g., myotubes).

The innovation has demonstrated that nanoscale features govern the differentiation of individual cells (e.g., myoblasts) into differentiated cells (e.g., myocytes) whereas microscale alignment cues orchestrate fusion of multiple cells such as myocytes into multinucleated fibers such as myotubes. The innovation presents the importance of multiscale hierarchy in enhancing coordinated tissue regeneration.

To accomplish the foregoing and related ends, certain illustrative aspects of the innovation are described herein in connection with the following description and the annexed drawings. These aspects are indicative, however, of but a few of the various ways in which the principles of the innovation can be employed and the subject innovation is intended to include all such aspects and their equivalents. Other advantages and novel features of the innovation will become apparent from the following detailed description of the innovation when considered in conjunction with the drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a schematic diagram of the hierarchical structure in accordance with aspects of the innovation.

FIG. 2 illustrates a flow chart detailing the method of fabricating the hierarchical structure in accordance with aspects of the innovation.

FIGS. 3A, 3B, and 3C illustrate SEM micrographs of carbon nanotubes attached on commercial cellular foam using one of the processes in accordance with aspects of the innovation: (a) low magnification image, (b) higher magnification image of ligaments, and (c) image showing dense growth possible in some areas even inside a pore.

FIGS. 4A, 4B, and 4C illustrate SEM micrographs of carbon nanotubes attached on commercial reticulated carbon foam using the same process at (a) 50× magnification, (b) 1,000× magnification, and (c) 5,000× magnification.

FIGS. 5A and 5B illustrate a schematic profile of the surface of a pore showing that compared to a normal surface (a), the hierarchical surface with nanoscale attachments can accommodate significantly larger number of surface active nanoparticles as shown in (b). The increase in particle density can be controlled by density and length of nanotubes grafted on the surface.

FIG. 6 illustrates Electron Microscope images of Pd-NP (nanoparticle) structures in accordance with aspects of the innovation.

FIG. 7 illustrates SEM micrographs of silver nanoparticles attached to nanotubes that have been grafted on microcellular foam specimens.

FIG. 8 illustrates is an example of the particle distribution obtained for Ag nanoparticles obtained at two reduction temperatures.

FIGS. 9A and 9B illustrate shows how the hierarchical structures in accordance with aspects of the innovation are effective in bacteria removal from water. The dark spots are bacterial colonies formed in lake water when (a) untreated (b) treated with 4 mm×4 mm×2 mm samples of AgNP-CNT/Foam (as shown in FIG. 7).

FIGS. 10A and 10B illustrate schematics of example techniques by which the nanoparticle-nanotube activated porous material can be used to remove contaminants from liquids.

FIG. 11 illustrates ultrahigh surface area hierarchical substrates in accordance with aspects of the subject innovation, which can be very useful as supports for nanocatalysts, sensors and advanced composites.

FIG. 12 illustrates a method of applying an oxide coating to a nanotube-grafted substrate in accordance with aspects of the subject innovation.

FIG. 13 illustrates images of a carbon nanotube (CNT) hierarchical surface before and after silica coating by techniques of the subject innovation, and their respective responses to a drop of water.

FIG. 14 illustrates images of a water droplet on an initial CNT forest, after plasma treatment, and after reheating in air.

FIG. 15 illustrates microstructures of CNT-HOPG (highly oriented pyrolytic graphite), oxygen treated CNT-HOPG (O-CNT-HOPG) surface, and the same surface after air annealing, along with contact angles of water on each.

FIG. 16 illustrates C 1s scans of CNT-HOPG, O-CNT-HOPG, and air annealed-O-CNT-HOPG.

FIG. 17 illustrates the microstructure of silica coated CNT-HOPG surfaces (silica-CNT-HOPG).

FIG. 18 illustrates STEM images of both as-grown CNT and silica-CNT.

FIG. 19 illustrates XRD patterns of as-grown CNT and silica-CNT.

FIG. 20 illustrates the microstructure and contact angle of a silica-CNT-HOPG sample from experiments discussed herein.

FIG. 21 illustrates the average ΔP/L for a given volumetric flux over three samples each for bare reticulated carbon foam (RVC foam), CNT grafted RVC foam (CNT-RVC), and silica coated CNT-RVC (silica-CNT-RVC).

FIG. 22A illustrates characterization of pristine and nano-functionalized foam scaffolds in accordance with an aspect of the innovation. FIG. 22B are SEM images showing the effect of surface nano-functionalization of carbon foams with CNT and Si-CNT on the micro- and nanoscale morphology according to an embodiment of the innovation.

FIGS. 23A-23E depict effects of surface nano-functionalization on myoblast morphology, metabolic activity and differentiation into myocytes according to embodiments of the innovation.

FIGS. 24A-24C depict characterizations of aligned pristine and nano-functionalized carbon fiber mats in embodiments of the innovation.

FIGS. 25A and 25B depict effects of carbon nanotube functionalization on myoblast morphology and metabolic activity in embodiments of the innovation.

FIGS. 26A-26F illustrate effects of fiber alignment on myoblast differentiation into multinucleated myotubes for embodiments of the innovation

FIG. 27 provides a detailed multiscale architecture of nano-functionalized foams according to an aspect of the innovation.

FIGS. 28A and 28B are SEM images in accordance with an implementation of the innovation.

FIGS. 29A-29C are SEM images in accordance with an implementation of the innovation.

FIGS. 30A-30C illustrates representative SEM images in an embodiment of the innovation.

FIG. 31 depicts immunofluorescence images of MHC-stained cells in an embodiment of the innovation.

FIG. 32 provides an example embodiment method in accordance with an implementation of the innovation.

### DETAILED DESCRIPTION

To accomplish the foregoing and related ends, certain illustrative aspects of the innovation are described herein in connection with the following description and the annexed drawings. These aspects are indicative, however, of but a few of the various ways in which the principles of the innovation can be employed and the subject innovation is intended to include all such aspects and their equivalents. Other advantages and novel features of the innovation will become apparent from the following detailed description of the innovation when considered in conjunction with the drawings.

Embodiments of the subject innovation can be related to a hierarchical structure formed on a substrate that increases the specific surface area of the starting substrate by several orders of magnitude. The hierarchical structure of the subject innovation is referred to as an ultrahigh surface area material in that the surface area of the structure is at least several 100 times or more compared with that of the original substrate. The structure can comprise a solid substrate and a plurality of nanoscale attachments, wherein the substrate can be modified to ensure durability of the nanoscale attachments. The modified substrate can include an intermediate layer onto which at least one plurality of nanoscale attachments is attached. While the surface area of a solid substrate can be increased to a certain extent by other means such as porosity, there is a practical limit to such increase, because porosity makes the solid structurally weaker. Embodiments of the subject innovation can provide hierarchical structures wherein nanotubes can be attached on the solid, thereby providing orders of magnitude increase in surface area without compromising the strength or structural integrity, resulting in better performance while maintaining robustness.

Multi-scale hierarchical structures of the subject innovation can include solids that contain components characterized by different length scales. For example, nanoscale attachments according to the subject innovation can be strongly tethered to solid substrates, which are not characterized as nanoscale, such that the nanostructures do not detach from the parent substrate under service conditions. These structures can be suitable for anchoring nanoparticles, such as nanometals having known surface-active properties useful in non-limiting examples such as catalysis, sensing, hydrogen storage, electrochemical activity, cell interaction, or antimicrobial behavior.

Previously, nanomaterials with surfaces exposed to service environment have been mainly in the form of loose particles or dust that gets leached or dispersed into the environment (i.e. air, water, soil, or body) where used; thereby making nanomaterials expensive and risky to use. For example, previous investigators have entrapped nanoparticles in porous materials; however, the bonding between the nanotubes/nanoparticles has not been strong enough to effectively restrain nanoparticles to prevent loss or leaching.

Therefore, in conventional approaches, such nanoparticles having beneficial properties could not be used practically due to problems of retention of nanoparticles onto structure substrates leading to problems such as (i) loose nanoparticles clustering together during transportation and/or storage; (ii) nanoparticles escaping into the environment, thus requiring replenishment, i.e., redeposition; (iii) nanoparticles escaping into the environment thus causing potential detrimental effects on the ecosystem or other environmental concerns; and (iv) nanoparticles escaping into the environment thus causing unknown health risks to population due to nanoparticle exposure.

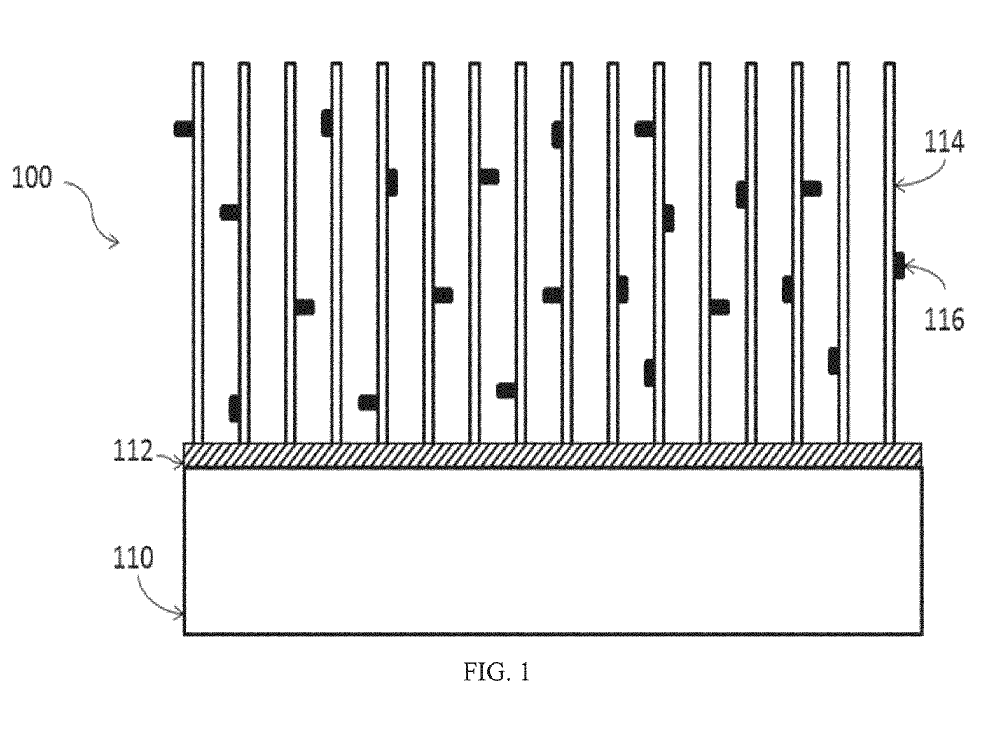

FIG. 1 is a schematic diagram of the hierarchical structures according to aspects of the subject innovation. Hierarchical structure 100 can comprise a solid substrate 110. The substrate can be modified or coated with chemicals, ions, plasma and/or heat to form an intermediate layer 112. To form the intermediate layer, one or more of the following non-limiting techniques may be used: (a) wet chemistry, (b) chemical vapor deposition, (c) plasma deposition, (d) atomic layer deposition, (e) physical vapor deposition, (f) controlled environment heating, or combinations thereof.

Further, nanoscale attachments such as nanotubes 114, nanoparticles 116, or combinations thereof can then be deposited or grown onto the intermediate layer 112 using liquid phase and/or vapor phase techniques involving one or more of (a) wet chemistry, (b) chemical vapor deposition, (c) plasma deposition, (d) atomic layer deposition, (e) physical vapor deposition, (f) controlled environment heating, or combinations thereof.

Different types of solids have been developed over the years for use in catalysts, sensors, filters, and sieves. Substrates 110 suitable for use in the subject innovation include both compact and porous solids. In at least one embodiment, the substrate may be inorganic or organic. In various embodiments, the substrate may include simple solids, fibers, foams, sponges, fabric, and/or paper. Substrates for use in one or more embodiments of the innovation may comprise carbon, oxides, polymers, or combinations or composites thereof. The substrate geometry may be simple or complex. Several grades of such material are already available from a wide range of commercial sources. These commercial substrates or other substrates can be advantageously engineered in the subject innovation to result in configurations wherein nanoparticles can be directly anchored to relatively flat or uneven porous solids; or alternatively, nanoparticles can be attached onto nanotubes that are tethered to the substrate; or combinations thereof.

In at least one embodiment of the subject innovation, the substrate can be highly oriented pyrolytic graphite (HOPG). In these or further embodiments of the subject innovation, the base structure can include microcellular or reticulate foam, an open cell structure, with porosities ranging from about 68% to about 94% and further characterized as having a high surface area.

Nanomaterials, materials having one or more dimensions less than 100 nm, are known for many beneficial properties. Nanomaterials may be referred to interchangeably herein as nanoscale attachments or nanoscale components. In at least one embodiment of the subject innovation, advantages arise from the increased specific surface area (SSA) of the solid without loss in mechanical integrity. Applications using structures comprising nanoscale attachments according to the subject innovation in at least one embodiment are directed to surface activity applications including: (i) catalysts for environmental purification using non-limiting examples of nanomaterials such as Pd, Ni, etc.; (ii) electrochemical activities such as charge storage and sensing using non-limiting examples of nanomaterials such as Pd, Ag, etc.; (iii) hydrogen storage and transport activities using non-limiting examples of carbon nanotubes (CNT) and Pd, etc.; (iv) antimicrobial activity using non-limiting examples of nanomaterials such as Ag, etc.; and (v) biological interactions such as cell scaffolding with non-limiting examples of CNT and/or anti-bacterial coatings using silver nanoparticles, etc. Using the advantageous structures comprising nanoscale attachments according to the subject innovation can result in time savings due to increased activity, space and weight savings due to miniaturization of the active device, and cost savings, especially for those devices utilizing precious metals.

In at least one embodiment of the subject innovation, the nanoscale attachments are nanotubes 114. In some embodiments, nanotubes according to the subject innovation may be carbon nanotubes (CNT), although in other embodiments, differing nanotubes can be used. In one embodiment, the carbon nanotube may be a single-walled carbon nanotube (SWCNT), a multi-walled carbon nanotube (MWCNT), or a combination thereof. In at least one embodiment of the subject innovation, CNTs can be grafted onto the base support. In yet another at least one embodiment of the subject innovation, the nanoscale attachments can be nanoparticles 116, which may include non-limiting examples such as iron nanoparticles (also referred to as FeNP), palladium nanoparticles (PdNP), silver nanoparticles (AgNP), or other nanoparticles. In at least one embodiment of the subject innovation, the nanoscale attachments can include nanoparticles attached onto nanotubes. In yet another embodiment, the nanoscale attachments can be further modified with chemicals or ions for fluid permeation and wettability.

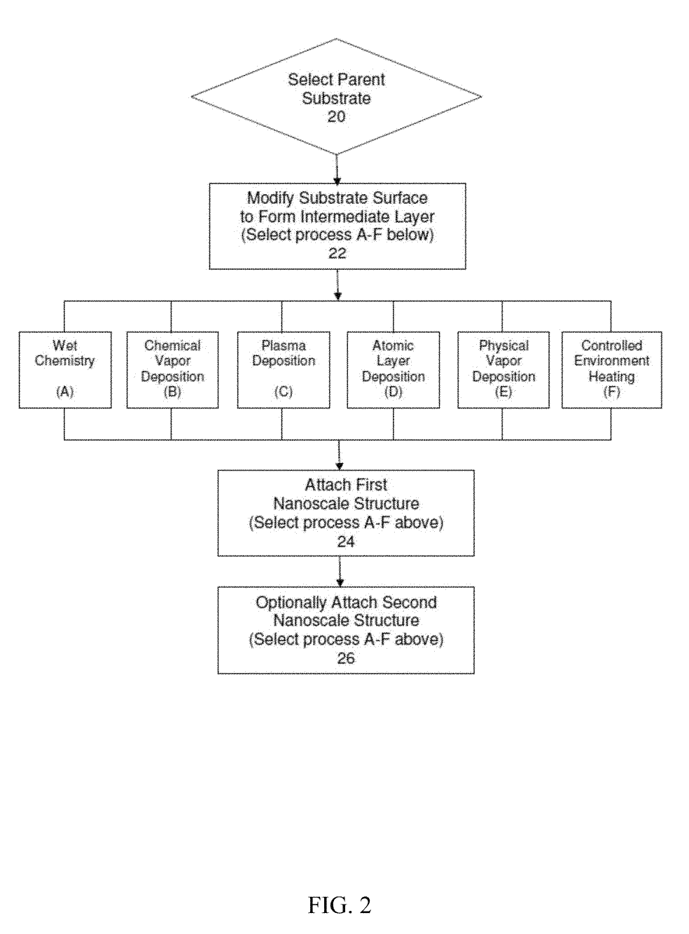

In at least one embodiment of the subject innovation, the nanoscale component(s) of selected metals can be attached to the substrate, wherein the substrate can be first modified at the surface to include an intermediate layer. The hierarchical structure of the subject innovation can be created by attachment of nanotubes onto substrates (e.g., commercially available substrates, etc.), which have been modified to include an intermediate layer. Strong attachment of the nanotubes and/or nanoparticles can be achieved by modifying the substrate to form an intermediate layer prior to attaching at least one plurality of nanoscale components. Examples of surface preparation/modification techniques according to the subject innovation can include, but are not limited to: cleaning and activation with chemical reagents, solvents, ions or plasma; deposition of nanoscale reactive layer by use of liquid precursors, molecular layering (atomic layer deposition), plasma deposition, chemical vapor deposition (CVD), or catalyst chemical vapor deposition (CCVD) methods; etc. In one or more embodiments of the subject innovation, surface modification techniques can be selected from the group (a) wet chemistry, (b) chemical vapor deposition, (c) plasma deposition, (d) atomic layer deposition, (e) physical vapor deposition, (f) controlled environment heating, or a combination thereof, as shown in FIG. 2. Technique selection can be tailored to the individual application, while also considering cost and quantity of the final commercial product. The surface preparation/attachment techniques according to the subject innovation can result in the formation of an intermediate layer 112.

In at least one embodiment of the subject innovation, the intermediate layer can be a nanometer-scale silica, also referred to as silicon dioxide or SiO₂, layer using microwave plasma deposition (MPD) of hexa-methyl-disiloxane (HMDSO) in O₂. Deposition of the oxide coating onto the substrate can be accomplished in three stages: (stage 1) introducing O₂ gas in to the microwave plasma chamber; (stage 2) subsequently flowing O₂ with HMDSO at increased microwave power to deposit oxide on the substrate surface; and (stage 3) introducing O₂ carrier gas into the chamber to stabilize the oxide coating at lower microwave power.

Silica thickness can be controlled by varying the coating time for stage 2 during MPD. The coating begins to manifest itself first as ‘island’ growth with heights of about 3 to 5 nm. The islands densely cover the surface of the substrate after about 30 seconds, and eventually coalesce by about 1 minute to form a uniform layer thickness of about 4 nm, with a surface roughness of less than 1 nm. The silica chemically bonds to the surface of the graphite substrate to form an interface of Si—C bonds. The intermediate layer of this particular embodiment, as defined herein, can include the Si—C bonds at the interface and silica deposited thereon.

After the intermediate oxide coating, or silica layer deposition as in this example, the sample can be transferred to the chemical vapor deposition (CVD) reactor for nanotube deposition by the floating catalyst method using a mixture of ferrocene and xylene. Longer CVD reaction times are possible and can result in further CNT growth and potential entanglement, which may be desired for some applications.

In at least one embodiment, the hierarchical structure including the substrate, the intermediate layer (oxide coating), and nanotubes deposited thereon can be further treated in order to include a second plurality of nanoscale attachments, for example, nanoparticles adhered onto the nanotubes. The nanoparticles, which may be gold, palladium, or silver, for example, can be formed by coating the hierarchical structure with precursor solution, reducing the metal containing precursors and subsequently heat treating in a controlled environment. These nanoparticles have the potential to act as catalysts, sensors, absorbents, or anti-microbial agents.

Similar or improved success may also be obtained by functionalizing the substrate to form an intermediate layer using one or more of the following techniques: (i) oxide coating using liquid precursors (e.g., commercial, etc.) followed by controlled thermal treatments, (ii) ultrathin coating of oxides such as aluminum oxide or silicon dioxide using atomic layer deposition, or (iii) intermediate layer of oxide using chemical vapor deposition (CVD). There are alternative means to prepare or modify the substrate surface in the practice of the subject innovation. For some substrates, as in one or more embodiments, the intermediate layer may be defined alternatively as an acid etched substrate surface.

Attachment of nanoscale components to the intermediate layer of hierarchical structures according to the subject innovation increases the available surface area of the overall solid by several orders of magnitude. This increased surface area can be used in multiple ways. One possibility is to use the bare nanotubes as “nano-radiators” that allow increased dissipation of heat or electric current across the surface. Another possibility is to use these hierarchical structures as a composite core material, or alternatively, as a template or scaffolding for cell growth. In one or more embodiments, the increased surface area can be utilized to support nanoparticles that can act as catalysts, sensors, or absorbents.

In at least one embodiment, the substrate with nanotubes, nanoparticles, or combinations thereof deposited thereon can be further functionalized for increased wettability or infiltration with other materials such as fluids, resins or energy storage (phase change) materials.

Strong attachment in a testing environment can be defined such that the nanoscale attachments do not get detached from the substrate before the substrate itself is internally damaged through the testing. As a practical matter, strong attachment ensures that desired performance of the hierarchical structure is met under use conditions.

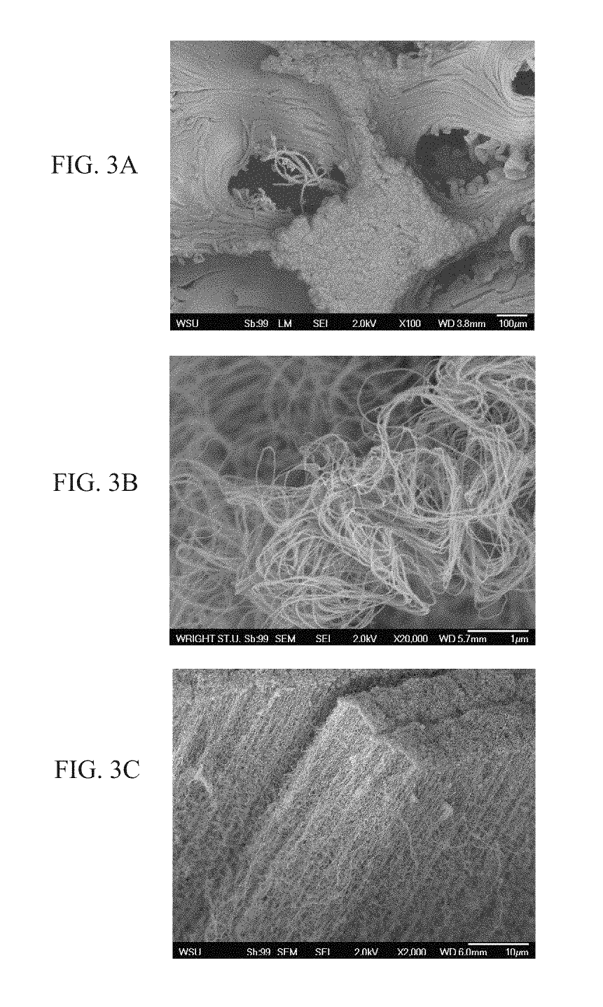

The initial success of nanotube and nanoparticle attachment can be observed by imaging with scanning electron microscopy (SEM), as seen in FIGS. 3, 4, 6 and 7. Measuring the average length, density and diameter of nanotubes provides an estimate of the increase in surface area. This number, along with a microscopic measurement of number of nanoparticles per unit length of nanotube, provides an estimate of the density of metal nanoparticles that can be packed on the surface. In one or more embodiments, over 7×10¹² nanoparticles/cm² (or 70,000 nanoparticles per square micron) could be accommodated on the surface using nanotube attachment as in FIG. 5B compared to 3×10¹⁰ nanoparticles/cm² (or 300 nanoparticles per square micron) on untreated surface similar to FIG. 5A.

To make direct determination of the specific surface area (SSA) of the substrate, the Brunauer-Emmett-Teller (BET) technique with nitrogen may be used. In one or more embodiments, the ultrahigh surface area hierarchical structures of the subject innovation are characterized by an increase in surface area by at least about 100-fold. In other words, the hierarchical structure can have a surface area about 100 times that of the original substrate. In other embodiments, the ultrahigh hierarchical structure can be characterized by having a specific surface area that is from at least about 100 to about 1000 times (10,000-100,000%) higher than the area of the starting substrate, while maintaining the same mechanical strength and minimal (less than 0.03 times or 3%) increase in weight.

In one or more embodiments, a starting foam substrate having an estimated specific surface area less than 0.02 m²/g could be modified with carbon nanotubes to create a hierarchical structure characterized by a surface area in the range of about 2 to about 4 m²/g.

In one or more embodiments, the hierarchical structures according to the subject innovation can be characterized by the nanoparticles having a controlled particle size distribution from about 3 nm to about 8 nm. In yet other embodiments of the subject innovation, the hierarchical structures can be characterized by the nanoparticles having a broad particle size distribution from about 5 nm to about 100 nm.

Qualitative failure analysis to determine the success of nanoscale attachment may be performed as follows. Samples broken at the edges can be analyzed microscopically to evaluate if the nanotube attachments, resembling nanotube ‘forests,’ are still attached. It has been observed in samples prepared according to the subject innovation that nanotubes remain attached, and fracture paths are indicated inside the graphite substrate itself rather than at the nanotube roots. This is defined as “strong bonding” of nanoscale attachments, when failure occurs in other parts of the substrate rather than at the intermediate layer wherein the nanoscale attachments are tethered to the substrate.

Another method useful in evaluating the success of nanotube attachment according to the subject innovation includes ultrasonication in water. The ultrasonication is believed to put large stresses at the base of the nanotube attachments, since their high aspect ratio would magnify any force at the tips caused by moving water. Despite that, failures observed indicate that the entire top layer of substrate peels off like a carpet; however, individual nanotubes (or nanoparticles attached to them) are not shed.

Yet another method to rapidly evaluate nanotube attachment is the Scotch® Tape test. Samples according to the subject innovation demonstrate that about 95% of the surface remains unchanged; in other words, nanoscale attachments are generally retained by the hierarchical structure. In the remaining about 5% area that does indicate loss of nanotubes, it has been observed that the entire outer substrate layer is removed like a carpet rather than as individual nanotubes. This thereby indicates failure occurring within the substrate itself, and is a further indicator of strong attachment of nanotubes to the substrate via the intermediate layer.

Samples according to the subject innovation have also been tested by rotation in water. These hierarchical structures have been attached to bottles filled with water and subjected to prolonged rotations for days and weeks at 32 revolutions per minute. No visible changes in nanotube or metal nanoparticle density after long exposures to rotation in water were observed using electron microscopy imaging techniques.

Shown in FIG. 2 is a flow chart of a method that can include the following steps in fabricating a multiscale hierarchical structure according to the subject innovation. While, for purposes of simplicity of explanation, the one or more methodologies shown herein, e.g., in the form of a flow chart, are shown and described as a series of acts, it is to be understood and appreciated that the subject innovation is not limited by the order of acts, as some acts may, in accordance with the innovation, occur in a different order and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with the innovation.

Step 20 includes selecting a substrate appropriate to the desired application. Step 22 includes modifying the substrate to form an intermediate layer using one or more surface modification techniques selected from the group (a) wet chemistry, (b) chemical vapor deposition, (c) plasma deposition, (d) atomic layer deposition, (e) physical vapor deposition, (f) controlled environment heating, or a combination thereof. Step 24 includes depositing or attaching at least one plurality of nanoscale attachments (nanotubes, nanoparticles, or combinations thereof) onto the intermediate layer, which may also be referred to as the coated substrate. The nanoscale attachments may be formed by using one or more techniques (a) through (f) described above or by combinations thereof. Step 26 includes optionally depositing or attaching a second plurality of nanoscale attachments (nanotubes, nanoparticles, or combinations thereof) onto the intermediate layer, the first plurality of nanoscale attachments, or to both the intermediate layer and the first plurality of nanoscale attachments. The nanoscale attachments may be formed by using one or more techniques (a) through (f) described above or by combinations thereof. Each of the steps 22, 24, and 26 may be optionally combined with heating in controlled environments such as in inert, oxidizing or reducing gases, or in vacuum as is known in the art.

In various aspects, embodiments of the subject innovation can be directed towards creation of hybrid hierarchical materials that consist of smaller nanoscale structures strongly tethered to larger solid substrates, and related methods of fabrication and use.

The hierarchical materials in accordance with aspects of the innovation are useful in applications such as, but not limited to, the following:

(i) thermal and charge dissipation structures made from carbon nanotubes attached to larger substrates: carbon nanotubes, having high electrical and thermal conductivity, can be used, for example, as nano-radiators for electronic cooling, electromagnetic interference shielding, encapsulation of energy storage materials, and other applications;

(ii) core structure for composites: the fractal geometry of the surface provides more intimate bonding with the matrix, thereby improving toughness and durability;

(iii) scaffolding for biological growth;

(iv) catalysts for electrochemical reaction, hydrogen storage and water de-contamination, utilizing nanoparticles such as palladium;

(v) antimicrobial agents and sensors, utilizing nanoparticles such as gold and silver. Solid silver attached to an anchor has several advantages over chlorinated compounds and other common anti-bacterial chemicals that are directly dispersed in the water, spread in the environment, and consumed in food. In addition to other side effects, these chemicals appear to increase the development of antibiotic-resistant strains of bacteria. Solids of the subject innovation can significantly reduce the need for antibacterial chemicals in the water and directly address some of the above issues.

Multiscale hierarchical extracellular matrix architecture at the macro-, micro- and nanoscale along with biomimetic cell-extracellular matrix interactions are some of the most important features to be considered for the development of functional muscle tissue which itself possesses multiscale hierarchy. Cell-interactive extracellular matrix-mimicking materials can facilitate essential cellular processes for normal muscle growth, development and regeneration. Specifically, a combination of biochemical and biophysical stimuli are critical for guiding appropriate orientation of progenitor cells to promote formation of aligned and well-organized muscle bundles. These biophysical stimuli may include nano-roughness, topographical guidance and electrically conductive surface.

Surface topography, especially nanoscale surface roughness can be an important feature in improving cell-scaffold interaction and cell adhesion. Since skeletal muscle tissue is electrically excitable, electroactive materials allow local delivery of electrical stimulus to enhance cell proliferation, differentiation and tissue regeneration.

Apart from nanoscale features, many native tissues, including muscles, display multiscale hierarchical structure responsible for their unique architecture and function. In the case of muscle tissue, submicron scale myofibrils constitute the basic structural units, which further organize into muscle fibers (10-80 microns in diameter), which then bundle together to form muscle fasciculus and eventually, a functional muscle. Thus, to enhance regeneration of a functional skeletal muscle, it is desirable to recreate multiscale hierarchical scaffolds that are composed of repeating small structural units to create a three-dimensional architecture.

Thus, for functional tissue regeneration, including skeletal muscle, it is advantageous to build multiscale hierarchical scaffolds that provide contact guidance to cells (e.g., myoblasts) through nano-roughness, provide alignment cues at microscale and allow cell infiltration as well as nutrient exchange.

Scaffolds possessing each of these individual features have been built; however, combining multiple such features into a single scaffold and a comprehensive understanding of their interplay in generation of a functional tissue has not been achieved in the prior art. This can be an especially daunting task given that all the materials may not be processed into controlled hierarchical architecture with simultaneous control over their physicochemical properties.

According to an aspect of the innovation, the scaffold may promote differentiation of various cell types. Suitable cell types include, but are not limited to, muscle, neuronal, and/or cardiac cells. In one embodiment, the scaffold according to the innovation may promote regenerative stimulation in cultured stem cells. In one embodiment, the scaffold may promote differentiation of progenitor cells, including myoblasts. In one embodiment, the scaffold may be used for in vitro testing of various cell/tissue types.

According to an aspect of the present innovation, electrically conductive carbon-based scaffolds can be processed to possess multiscale hierarchy along with macro-porous structure and different geometries.

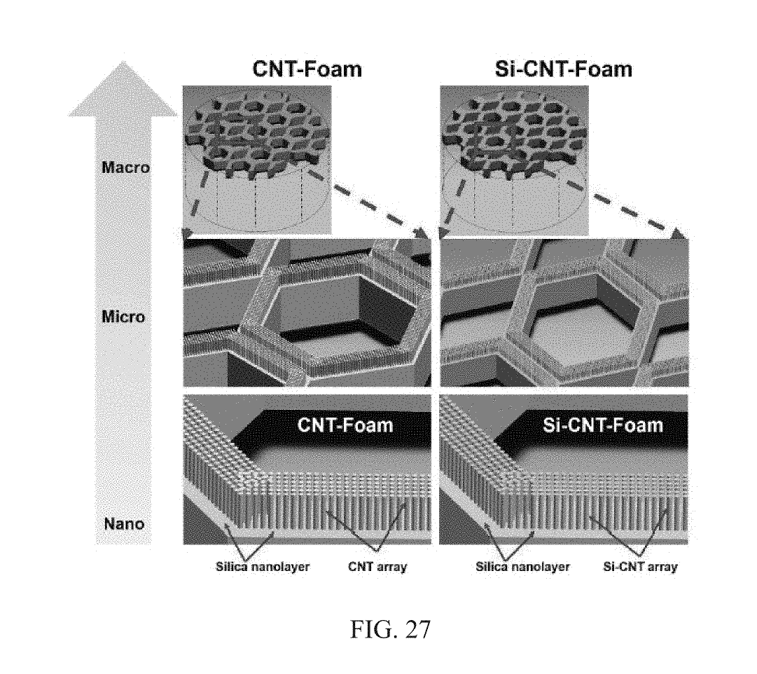

In aspects of the innovation, the nano-functionalization of foam scaffolds affects nano-roughness and wettability. Hence, multiscale hierarchical carbon foams with interconnected microporous structure may be nano-functionalized with carpets of carbon nanotube to modulate nano-roughness and wettability. Surface functionalization with nanoscale features alters wettability and nano-roughness, which, in turn, may affect cell adhesion, spreading and differentiation. Detailed multiscale architecture of nano-functionalized foams is depicted in FIG. 27.

The wettability of Pristine-Foam and Carbon Nanotube-Foam was measured by contact angle of water droplet on the scaffold surface. Carbon nanotube grafting on the foam surface increased the contact angle from 65-75° to 160° (Table 1), making the surfaces less wettable.

Table 1 demonstrates the effect of surface nano-functionalization on contact angle of different foams. The contact angle of foams increased with carbon nanotube grafting, while subsequent coating of Si on carbon nanotube-coated foams restored the contact angle similar to pristine-foams.

TABLE 1

| | Contact |
|---|---|
| Composition | angle (°) |
| Pristine-Foam | 65-75° |
| CNT-Foam | 160° |
| Si-CNT Foam | 66.5° |

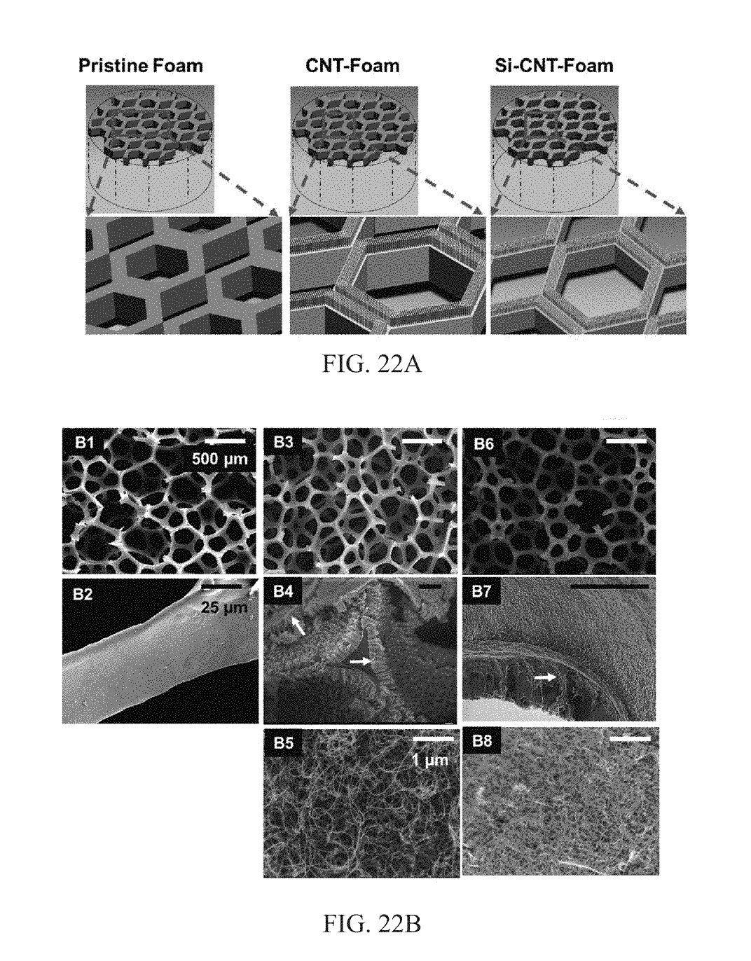

Nano-roughness of Carbon Nanotube-Foam can be characterized using scanning electron microscopy (hereafter “SEM”). FIG. 22B shows SEM images of Pristine-Foam and Carbon Nanotube-Foam. As shown in FIGS. 22B(B1) and 22B(B3), Pristine-Foam and Carbon Nanotube-Foam exhibited interconnected porous structure with overall similar pore size/porosity. Carbon nanotube carpet of about 20 lm thickness was found to grow perpendicular to the surface of pristine-foam scaffolds (FIG. 22B(B4), white arrows). Successful carbon nanotube functionalization was further confirmed by high magnification (20,000×) images of the walls of functionalized foam showing uniform fibrous carpet of carbon nanotubes rendering nanoscale roughness to these surfaces (FIG. 22B(B5)) compared to smooth surface observed for pristine-foam scaffolds (FIG. 22B(B2)). Thus, carbon nanotube functionalization increased the contact angle rendering the surface less wettable and also increased the nano-roughness of Carbon Nanotube-Foams. These results indicate that carbon nanotube functionalization affected multiple factors, such as surface wettability and nano-roughness that would further influence the cell-scaffold interaction.

Turning to FIG. 22, Characterization of pristine and nano-functionalized foam scaffolds according to aspects of the innovation are shown. (A) Schematic of hierarchical multiscale architecture of carbon foams functionalized with carbon nanotubes (carbon nanotube) and silica-carbon nanotube (Si-carbon nanotube); (B) Effect of surface nano-functionalization of carbon foams with carbon nanotube and Si-carbon nanotube on the micro- and nanoscale morphology by SEM; Low magnification images showing highly interconnected porous structure of Pristine-Foam (B1), Carbon Nanotube-Foam (B3) and Si-Carbon Nanotube-Foam (B6); High magnification images showing smooth surface of Pristine-Foams (B2) while carbon nanotube (B4) and Si-carbon nanotube (B7) functionalization conferred nano-roughness (depicted by white arrows); carbon nanotube (B5) and Si-carbon nanotube coating (B8) at 20,000× magnification showing rough nanofibrous structure. Scale bar in B1, B3 and B6 represents 500 μm, scale bar in B2, B4, and B7 represents 25 μm and scale bar in B5 and B8 represents 1 μm. Carbon nanotube structures and foam architecture shown in the schematics are only for the demonstration of the concept—schematics not to scale.

In another embodiment of the innovation, Carbon Nanotube-Foams may be further functionalized with amorphous silica (SiOx) (referred as Si-Carbon Nanotube-Foam hereafter) layer. An aspect of the innovation is that immobilized carbon nanotubes/Si-carbon nanotubes will exhibit excellent cytocompatibility and will promote myoblast growth and differentiation into myocytes by virtue of structural cues such as and macro-porous scaffold architecture as well as other factors such as nano-roughness, wettability, conductive surfaces. Silica is a hydrophilic, inorganic, bio-resorbable and biocompatible material that has been widely investigated in tissue engineering, specifically in bone regeneration. Silica functionalization on carbon nanotube may change the combination's wettability. However, the silica functionalization also maintains the same nano-roughness as that of Carbon Nanotube-Foam, thus decoupling the effect of wettability and nano-roughness on cell adhesion/spreading.

In one embodiment, an increase in the percentage of Si atoms from 1.38% (Carbon Nanotube-Foam) to 4.32% (Si-Carbon Nanotube-Foam) can be verified by elemental analysis of Si-Carbon Nanotube-Foams showing successful nano-functionalization of carbon nanotubes with Si (Table 2).

Table 2 shows energy-dispersive X-ray spectroscopy (EDS) analysis of Pristine, Carbon Nanotube and Si-Carbon Nanotube-Foams according to an embodiment showing atomic percentage of C, O, Si and Fe based on K (innermost) shell. “ND” (not detectable) indicates that the values for these elements were below detection limits of the instrument.

TABLE 2

| | Pristine- | | Si-CNT- |
|---|---|---|---|
| | Foam | CNT-Foam | Foam |
| Element | (At %) | (At %) | (At %) |
| C | 91.81 | 95.82 | 86.79 |
| O | 68.19 | 62.81 | 67.81 |
| Si | ND | 61.38 | 64.32 |
| Fe | ND | 60.19 | 61.68 |

It can also be seen that the water contact angle of the carbon nanotube carpets decreased from 160.3° (Carbon Nanotube-Foam) to 66.5° (Si-Carbon Nanotube-Foam) (Table 1) implying that Si-Carbon Nanotube-Foam is no longer hydrophobic despite the presence of carbon nanotube carpet morphology.

Characterization of the nano-roughness of Si-Carbon Nanotube-Foam may use SEM. As shown in FIG. 22B(B6), Si-Carbon Nanotube-Foam exhibited interconnected porous structure with similar pore size as Pristine-Foam and Carbon Nanotube-Foam. Si-carbon nanotube coating was found to be perpendicular to the surface of the scaffold (FIG. 22B(B7), white arrow) similar to that of Carbon Nanotube-Foam (FIG. 22B(B4)). As shown in FIG. 22B(B8), uniform fibrous carpet of Si-carbon nanotube conferred nano-roughness to the surface, similar to Carbon Nanotube-Foam (FIG. 22B(B5)). Si-Carbon Nanotube-Foam possesses comparable macro-porosity, contact angle and wettability to that of Pristine-Foam with added nano-roughness of Carbon Nanotube-Foam.

The aspect of the innovation's cytocompatibility as well as ability to promote cell adhesion, spreading, and differentiation may be assessed following the characterization of the Foam scaffolds.

One aspect of the innovation is that immobilization of carbon nanotube carpets on Pristine-Foams as being achieved through strong interaction with the underlying silica substrate nanolayer (FIG. 27), which is utilized to prevent shedding of individual carbon nanotubes and minimize cellular uptake. The physicochemical characterization of Pristine and Carbon Nanotube-Foams confirms that the interaction between carbon nanotube and under-lying silica nanolayer is much stronger than that between graphitic layers of the Pristine-Foam.

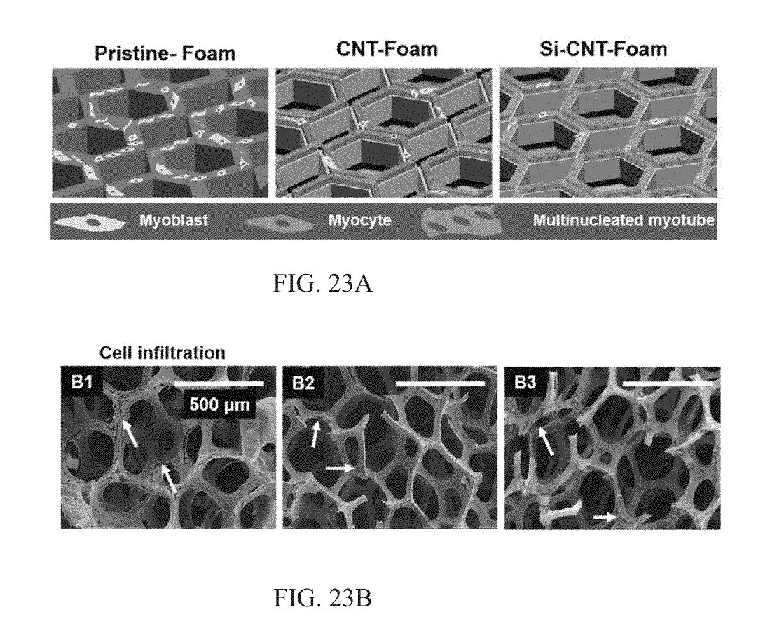

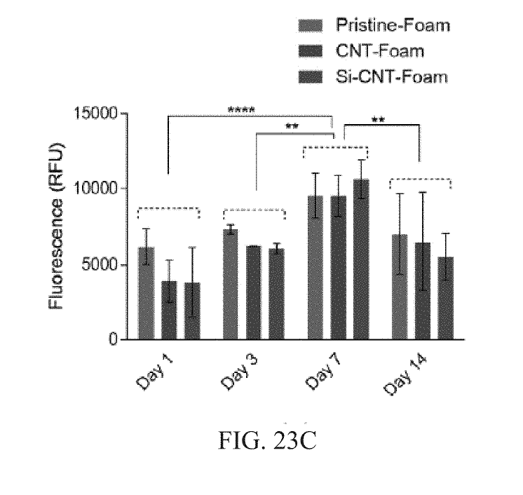

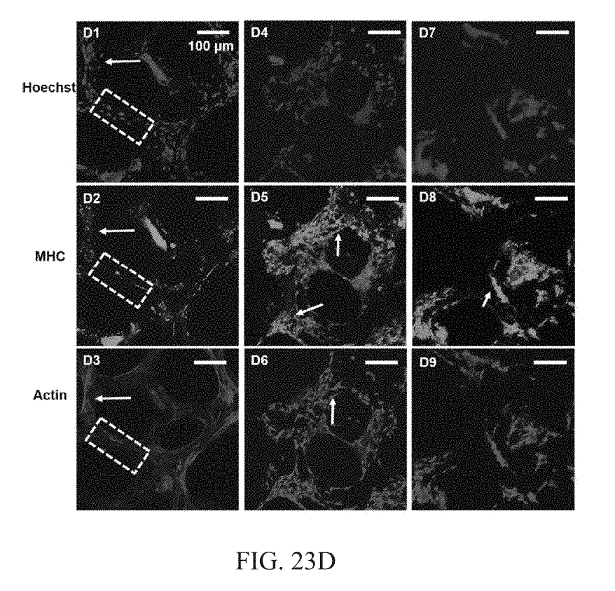

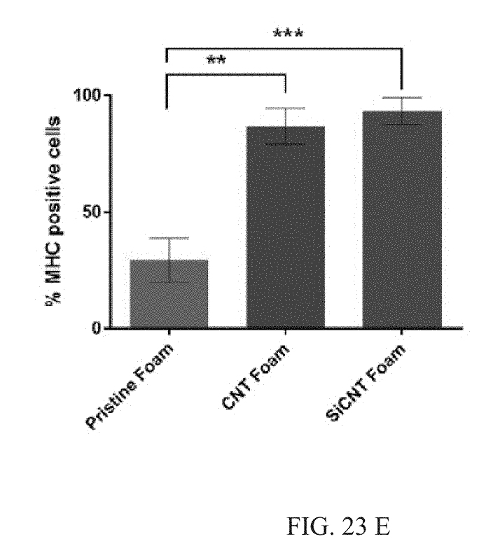

The innovation's aspects of cell adhesion, spreading and differentiation may be verified as in an example culturing of mouse myoblast (C2C12) cells on pristine and functionalized foam scaffolds (FIG. 23A).

FIG. 23 illustrates effects of surface nano-functionalization on myoblast morphology, metabolic activity and differentiation into myocytes in embodiments of the innovation. (A) A schematic showing C2C12 myoblast cells seeded onto pristine and functionalized foams; (B) Scanning electron microscopy images showing cell infiltration throughout the depth of Pristine-Foam (B1), Carbon Nanotube-Foam (B2), and Si-Carbon Nanotube-Foam (B3). Scale bar in B represents 500 lm. White arrows indicate cells adhering to the walls of foams; (C) Viability/metabolic activity of C2C12 cells by Alamar Blue_assay (n=3), Statistical significance by two-way ANOVA and Tukey post hoc test. **denotes p<0.005, ****denotes p<0.00005; (D) Confocal microscopy images of Pristine-Foam (D1, D2, D3), Carbon Nanotube-Foam (D4, D5, D6) and Si-Carbon Nanotube-Foam (D7, D8, D9) showing differentiation of C2C12 cells into myocytes as indicated by myosin heavy chain (MHC) staining (green, D2, D5, D8). The nuclei (blue, D1, D4, D7) and actin (red, D3, D6, D9) were stained with Hoechst and actin-phalloidin, respectively. Scale bar in D represents 100 μm. White arrows show areas showing MHC expressing cells while white box shows cells on Pristine-Foams positive for actin, but with no MHC expression. (E) Quantification of percentage MHC-positive cells after differentiation study (n>120 cells from at least 3 images and 3 different scaffolds). Statistical significance by oneway ANOVA and Tukey post hoc test. **denotes p<0.005, ***denotes p<0.0005.

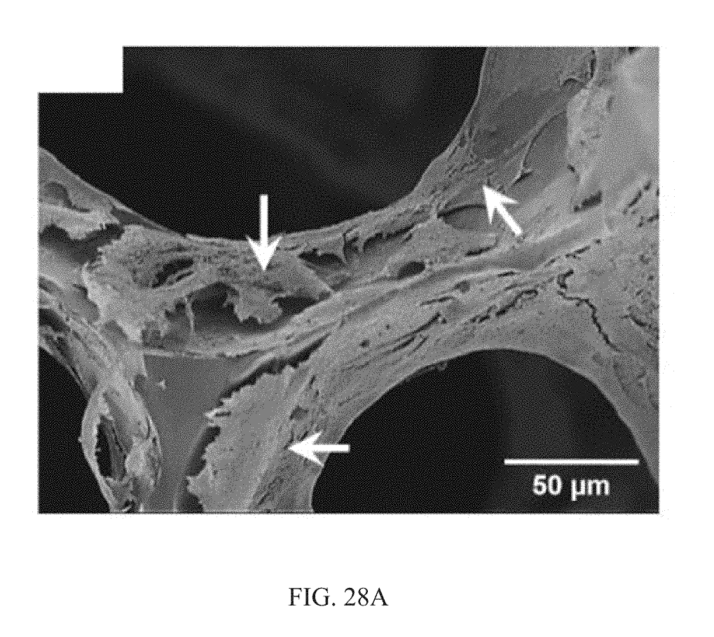

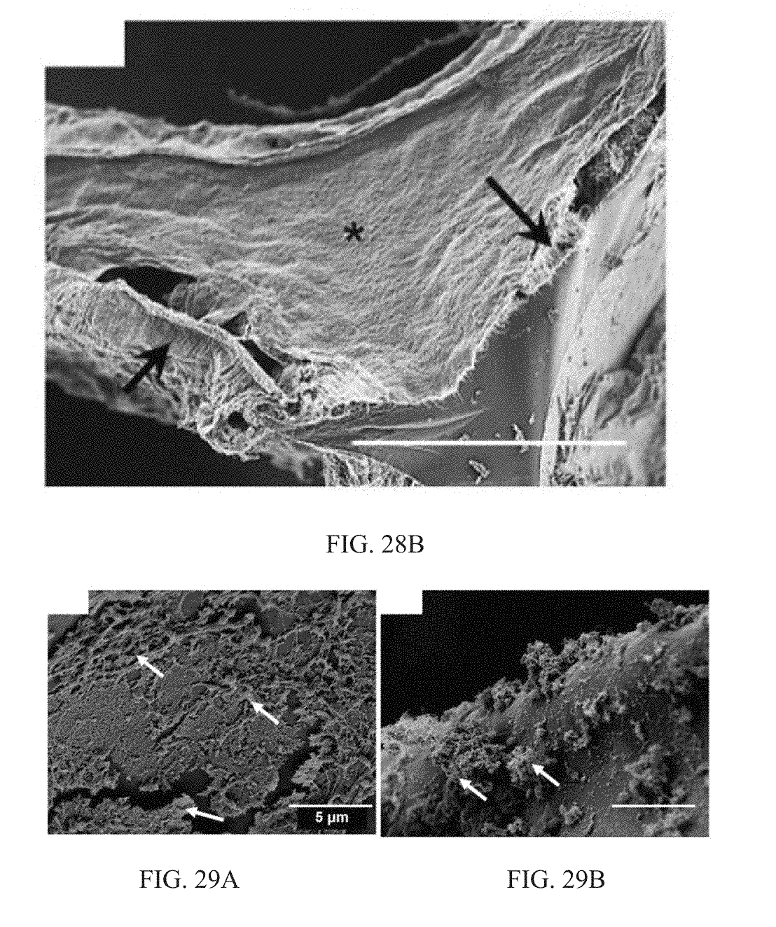

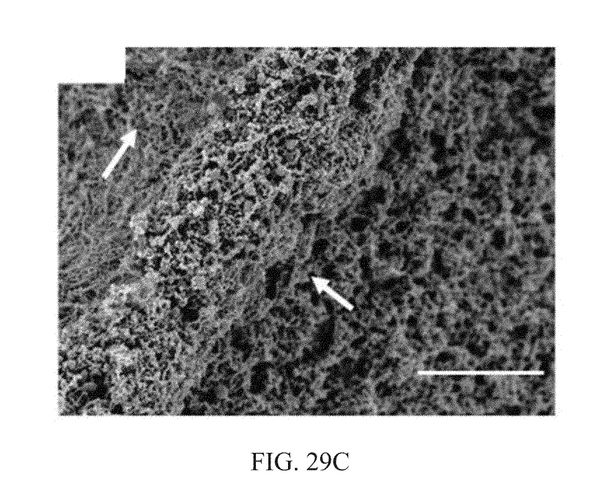

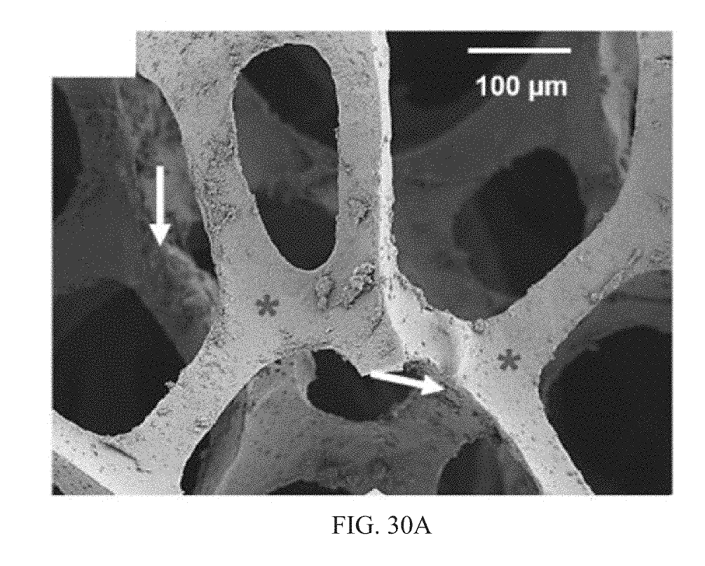

Infiltration of C2C12 cells into the scaffolds was studied after 21 days of culture using SEM. FIGS. 23B(B1), 23B(B2) and 23B(B3) indicate that cells were able to infiltrate and adhere (white arrows) uniformly to all the foam scaffolds. In case of Pristine-Foams (FIG. 23B(B1) and FIG. 28A), cells adhered to the foam wall, forming a uniform cell sheet over the surface, whereas the presence of nano-hair like carbon nanotubes in the other two scaffolds increased surface roughness and cell-scaffold interaction in the functionalized scaffolds as evident from the cells (pseudo colored in red, black asterisks) attached on to these hair-like projections (FIG. 28B, black arrows). Thus, SEM images indicate that nano-functionalization with carbon nanotube carpets enhanced cell-Foam interactions in the functionalized foam scaffolds.

In another aspect of the innovation, de novo matrix synthesis on biomaterial scaffolds is indicative of one of the essential steps in tissue regeneration. Production of functional tissue-mimetic structures can be facilitated through the synthesis of new extracellular matrix by the seeded cells along with biodegradation of the scaffolds. In the present example of mouse myoblast cells, Pristine-Foam, Carbon Nanotube-Foam and Si-Carbon Nanotube-Foam facilitated de novo extracellular matrix secretion on the surface of scaffolds as shown by white arrows in FIG. 23. All three foam scaffolds thus can be considered bioactive and facilitate cell adhesion, spreading and de novo matrix secretion.

Continuing with the example embodiment, the effect of surface nano-roughness and wettability on C2C12 cell adhesion and proliferation may be further investigated by determining the cells viability with, for example, Alamar Blue® assay (FIG. 23C). Alamar Blue® assay measures ability of metabolically active cells to reduce resazurin to fluorescent resorufin. All scaffolds supported growth of C2C12 as evident from the significant increase in their metabolic activity over a period of 7 days. It is noteworthy that differences between Pristine-Foams and Carbon Nanotube/Si-Carbon Nanotube-Foam were not statistically significant on any given day of culture, indicating that the immobilizing of carbon nanotube/Si-carbon nanotube carpets onto carbon foams did not significantly affect cell viability and growth. Thus, Carbon Nanotube/Si-Carbon Nanotube-Foams are found to be cytocompatible.

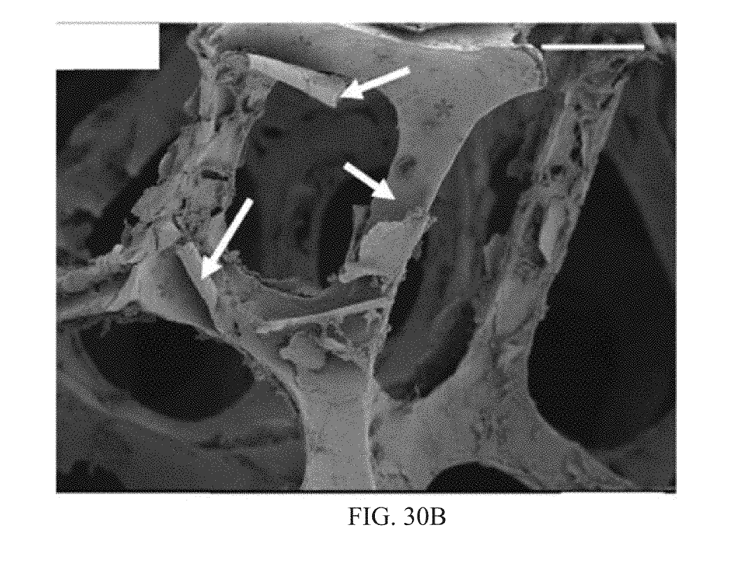

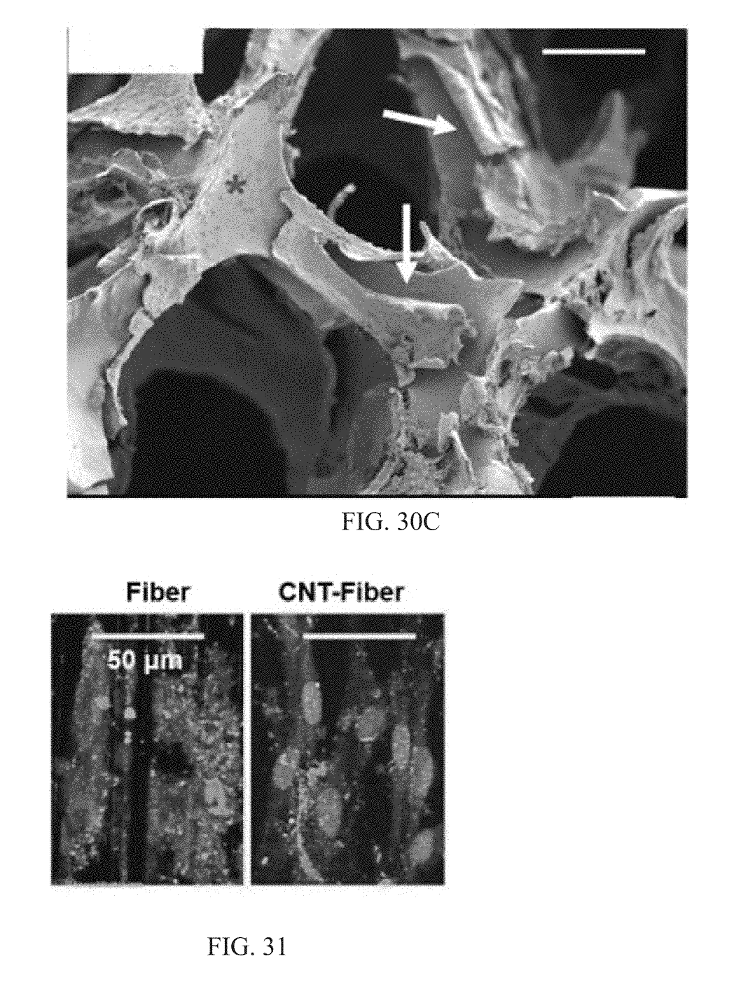

In an example embodiment, a decrease in the metabolic activity of cells on day 14 in all three scaffolds was observed. This may be attributed to two phenomena. First, the aspect of prevented individual nanotube shedding indicates that peeling of full cell sheets as opposed to individual carbon nanotubes may occur. This is observed in that confluent cell sheets peeled off from the carbon nanotube/Si-carbon nanotube-foam surfaces as shown in representative SEM images for carbon nanotube/Si-carbon nanotube-foam scaffolds (FIGS. 30A-30C, white arrows). SEM images also revealed comparatively smooth surfaces of carbon nanotube/Si-carbon nanotube-foam scaffolds after detachment of cell-sheets, similar to smooth surfaces observed in Pristine-Foams (FIG. 22B(B2) and FIG. 30A-30C, denoted by asterisks). These SEM images suggested that cell sheets peeled off along with the underlying carbon nanotube/Si-carbon nanotube carpets. Peeling off of carbon nanotube carpets as an entire layer from the foam surface has been attributed to the much stronger interaction between carbon nanotubes and underlying silica nano-layer than that between the graphitic layers in the underlying foam. SEM images in our study supports this conclusion and indicate that the cell sheets and carbon nanotube/Si-carbon nanotube carpets remained together during their detachment, further suggesting strong interaction of cells with underlying carbon nanotube/Si-carbon nanotube carpets. Such strong interaction between the myoblasts and carbon nanotube carpets may be beneficial in two ways. First, the carbon nanotube carpets can provide a mechanical support to the native myoblasts until they get anchored, oriented and differentiated into functional multinucleated myotubes. Second, this may also prevent disaggregation/detachment of individual carbon nanotubes and their uptake by the cells, thus obviating the cytotoxicity of carbon nanotubes.

The decrease in the metabolic activity at day 14 may also be due to a second phenomenon and aspect of the innovation: differentiation of myoblast cells into multinucleated myotubes. In an embodiment, the aspect of carbon nanotube and Si-carbon nanotube functionalization effect on myoblast differentiation can be seen. C2C12 cells may be cultured in growth media for 3 days, followed by an appropriate time, for example, 18 days in differentiation media. The cells may then be fixed and stained with Hoechst (nucleus, blue), myosin heavy chain (MHC, green) and phalloidin (actin, red). In an example embodiment, pristine-foam scaffolds displayed attachment and spreading of C2C12 cells on the foam walls as shown by nuclear and cytoskeletal actin staining (FIGS. 23D(D1) and 23D(D3), arrows). However, very few MHC-positive cells were observed on Pristine-Foam (FIG. 23D(D2), white box), indicating their limited ability to differentiate into MHC-positive myocytes. On the contrary, Carbon Nanotube-Foam (FIG. 23D(D4)-23D(D6)) and Si-Carbon Nanotube-Foam (FIG. 23D(D7)-23D(D9)) displayed presence of much higher number of MHC-positive cells, indicating that carbon nanotube/Si-carbon nanotube functionalization promoted differentiation of myoblasts into myocytes. Image analysis of the sample embodiment confirmed these results revealing significantly higher percentage of MHC-positive cells (about 90%) in Carbon Nanotube-Foam and Si-Carbon Nanotube-Foam compared to Pristine-Foam (only 30%, FIG. 23E). Such enhanced differentiation may be due to the nano-roughness provided by carbon nanotube/Si-carbon nanotube.

It should be noted that although Si coating on Carbon Nanotube-Foams restored their contact angle from 160° to 66° (similar to Pristine-Foams), altering the surface wettability, but did not inhibit the ability of Si-Carbon Nanotube-Foams to induce myoblast differentiation. On the contrary, both Carbon Nanotube/Si-Carbon Nanotube-Foam exhibited comparable percentage of MHC-positive cells despite significant differences in their contact angles (FIGS. 23D(D5) vs. 23D(D8) and 23E). This indicates that nano-roughness, which is a common feature of both Carbon Nanotube-Foam and Si-Carbon Nanotube-Foam rather than wettability plays the more important role in controlling myoblast differentiation into MHC-positive cells

In the example embodiment being discussed, nano-functionalized foams promoted myoblast differentiation into MHC-positive myocytes, but formation of multinucleated myotubes was not evident in any of the foam scaffolds. This may be due to the porous architecture of the foam scaffolds. Foams provide excellent porosity and surface area that enhanced myoblast cell proliferation and adhesion on all three scaffolds whereas carbon nanotube/Si-carbon nanotube functionalization provide additional structural cues such as nano-roughness and/or conductivity, which may further aid myoblast differentiation into MHC-positive cells. However, foams lack these alignment cues, which are also present in native skeletal muscle tissue. Although interconnected porous structure along with nanoscale surface functionalization led to myoblast differentiation into MHC-positive cells, it fell short of promoting fusion of these cells into multinucleated myotube-like structures, as otherwise provided by aspects of the innovation.

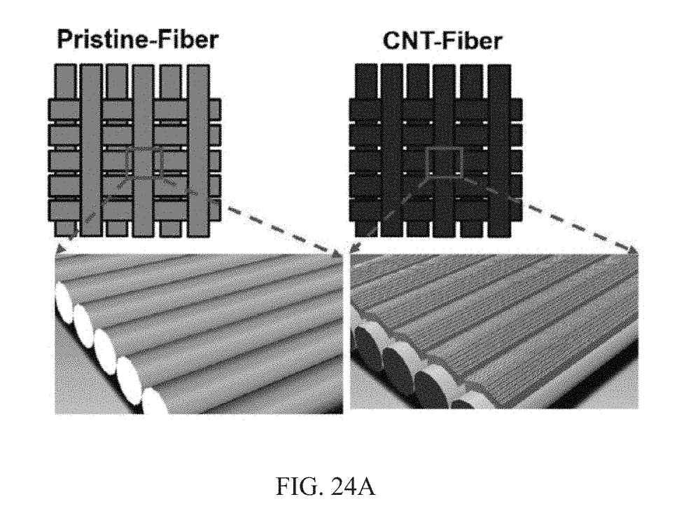

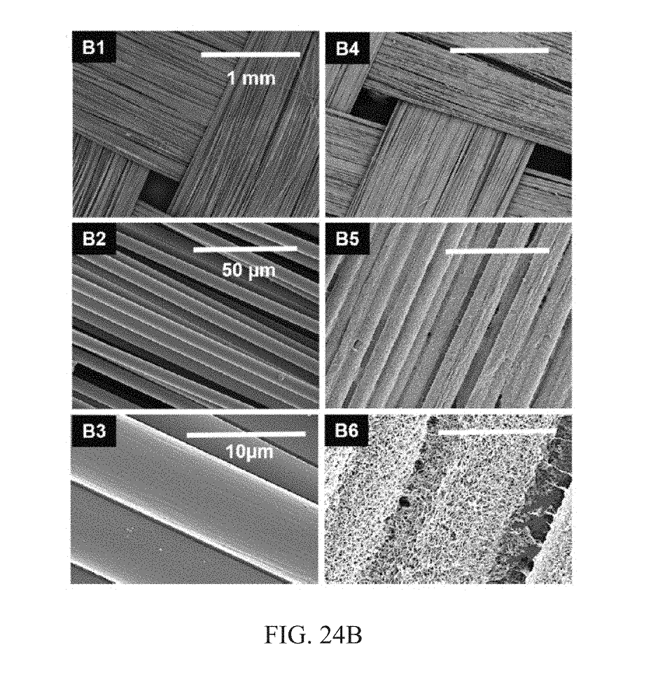

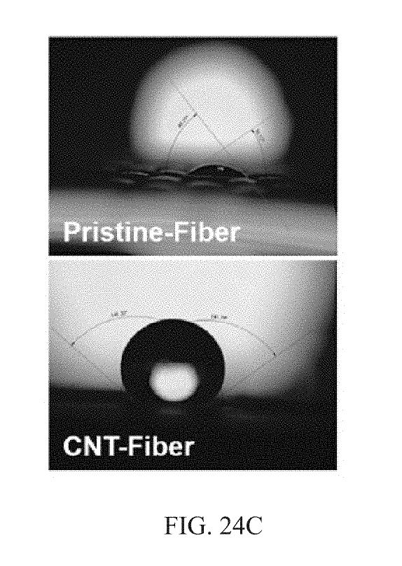

As skeletal muscle is a highly organized structure, myotube formation on surfaces without alignment cues may be immature; the innovation, therefore, functionalized aligned woven fibrous carbon mats with strongly anchored carbon nanotube carpets using the two-step technique discussed earlier (FIG. 24A).

FIG. 24. Characterization of aligned pristine and nano-functionalized carbon fiber mats is shown in embodiments of the innovation. (A) Schematic of hierarchical multiscale architecture of aligned non-functionalized carbon fibers (Pristine-Fibers) and functionalized Carbon Nanotube-Fibers; (B) Scanning electron microscopy images showing macro- to microscale morphology of Pristine-Fiber (B1-B3) and Carbon Nanotube-Fibers (B4-B6); carbon nanotube functionalization increased the surface roughness (B5 and B6) in contrast to the smooth surface of Pristine-Fibers (B2 and B3). Scale bar in B1 and B4 represents 1 mm, scale bar in B2 and B5 represents 50 μm and scale bar in B3 and B6 represents 10 μm; (C) carbon nanotube-functionalization increased contact angle and thus, decreased wettability of the carbon nanotube-fiber scaffolds as indicated in Table 3.

Woven fiber mats have multiscale hierarchy with microscale fibrils forming a horizontal multilayered strip. These strips are then interwoven with a plain weave construction as shown in the schematic (FIG. 24A). As evident from the study of Si-Carbon Nanotube-Foam, wettability did not show any significant effect on myoblast differentiation, therefore Si functionalization was not considered in an example study of an embodiment with carbon fiber mats. In this example embodiment, successful carbon nanotube functionalization of fibers was confirmed by SEM imaging. FIG. 24B shows SEM micrographs of pristine carbon fibers and carbon nanotube-coated carbon fibers (referred to as Carbon Nanotube-Fiber). After carbon nanotube grafting, the overall aligned scaffold architecture at macroscale remained unchanged while surface roughness was enhanced (FIG. 24B(B4-B6)) compared to smooth surface observed for Pristine-Fibers (FIG. 24B(B1-24B3)). In addition, some carbon nanotubes bridging two parallel carbon fibers were observed as indicated by red arrows in FIG. 24B(B6). Thus, in this example embodiment, carbon nanotube grafting conferred the nano-roughness to the surface along with interconnected grooves between parallel fibers without compromising the fiber alignment. In this example embodiment, carbon nanotube grafting also increased contact angle of the scaffolds similar to Carbon Nanotube-Foam (FIG. 24C and Table 3).

TABLE 3

| | Contact |
|---|---|
| Composition | angle (°) |
| Pristine-Fiber | 39.5 |
| CNT-Fiber | 141.3 |

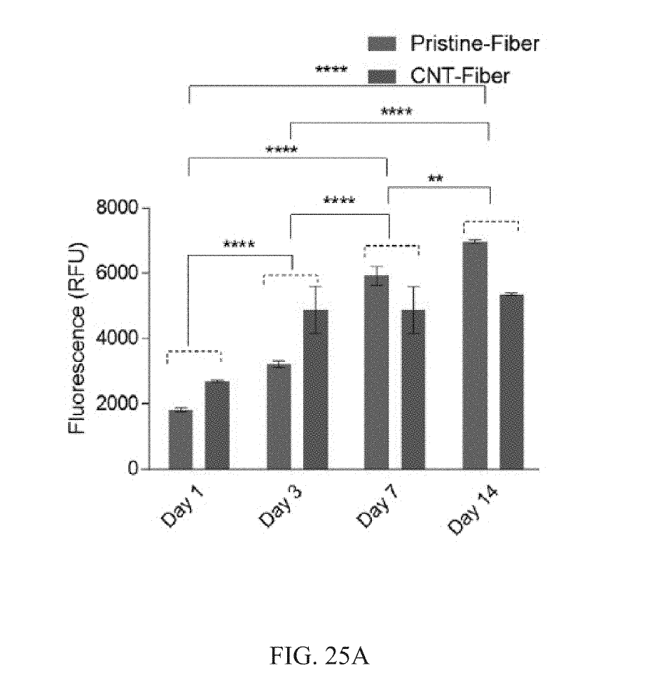

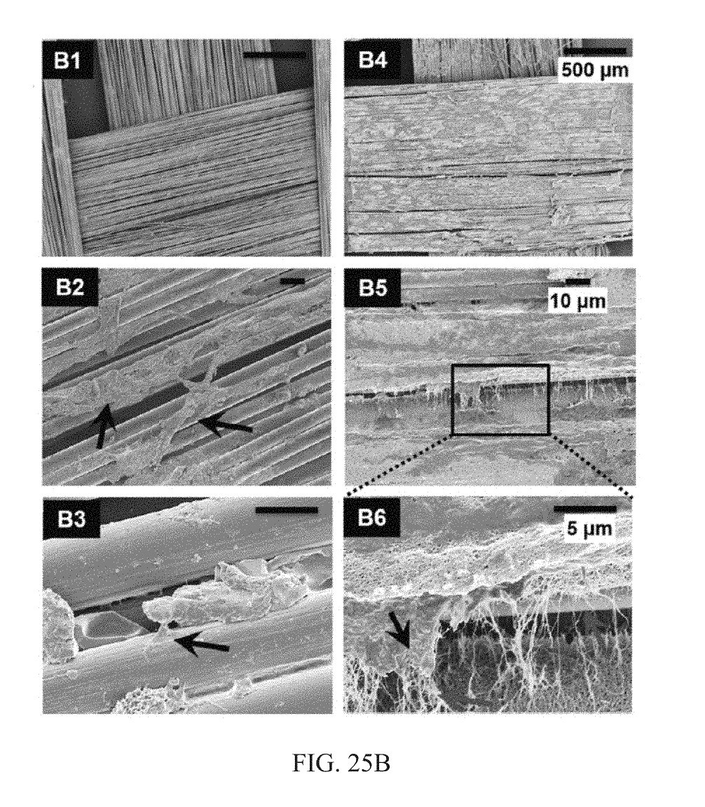

FIG. 25 illustrates the effects of carbon nanotube functionalization on myoblast morphology and metabolic activity in embodiments of the innovation. (A) Viability/metabolic activity of C2C12 cells on Pristine-Fiber and carbon nanotube-fiber mats by Alamar Blue® assay (n=3). Statistical significance by two-way ANOVA and Tukey post hoc test. **denotes p<0.005, ****denotes p<0.00005. (B) Scanning electron microscopy images showing C2C12 cell morphology and their interaction with the underlying pristine-fiber and carbon nanotube-fiber scaffolds. Significantly increased number of cells can be observed on the Carbon Nanotube-Fiber covering the entire area (B4). B5 and B6 show cell bodies (black arrows) interacting with the nanofibrous carbon nanotubes (red arrows) coated on carbon fibers. Scale bar in B1 and B4 represents 500 μm, scale bar in B2 and B5 represents 10 μm and scale bar in B3 and B6 represents 5 μm.

In another embodiment, C2C12 myoblast cells may be seeded on Pristine-Fiber and functionalized Carbon Nanotube-Fiber to elucidate the integrated effect of fiber alignment and carbon nanotube functionalization on cellular proliferation, alignment and differentiation. Metabolic activity of seeded cells were evaluated for 14 days using Alamar Blue® assay (FIG. 25A). Pristine-Fibers showed increased metabolic activity throughout 14 days while Carbon Nanotube-Fiber showed significantly higher metabolic activity than Pristine-Fibers until day 3 (p<0.05), after which Carbon Nanotube-Fiber reached confluence and no significant change was observed thereafter. As discussed above, such reduction in metabolic activity may be attributed to stimulation of myogenic differentiation by carbon nanotube carpet. While pristine-fiber scaffolds showed cell adhesion/spreading and facilitated formation of multicellular structures (black arrow in FIG. 25B(B2)), randomly orientated and less organized cell aggregates can also be observed. On the other hand, Carbon Nanotube-Fiber promoted formation of aligned multicellular structures completely covering the entire area of carbon nanotube-fiber scaffold (approx. 2.5 mm) as shown in FIGS. 25B(B4) and 25B(B5). Carbon nanotube-fiber scaffolds also promoted cell-material interaction to achieve significant contact adhesion as indicated by black arrows (cell bodies) and red arrows (carbon nanotubes) (FIG. 25B(B6)).

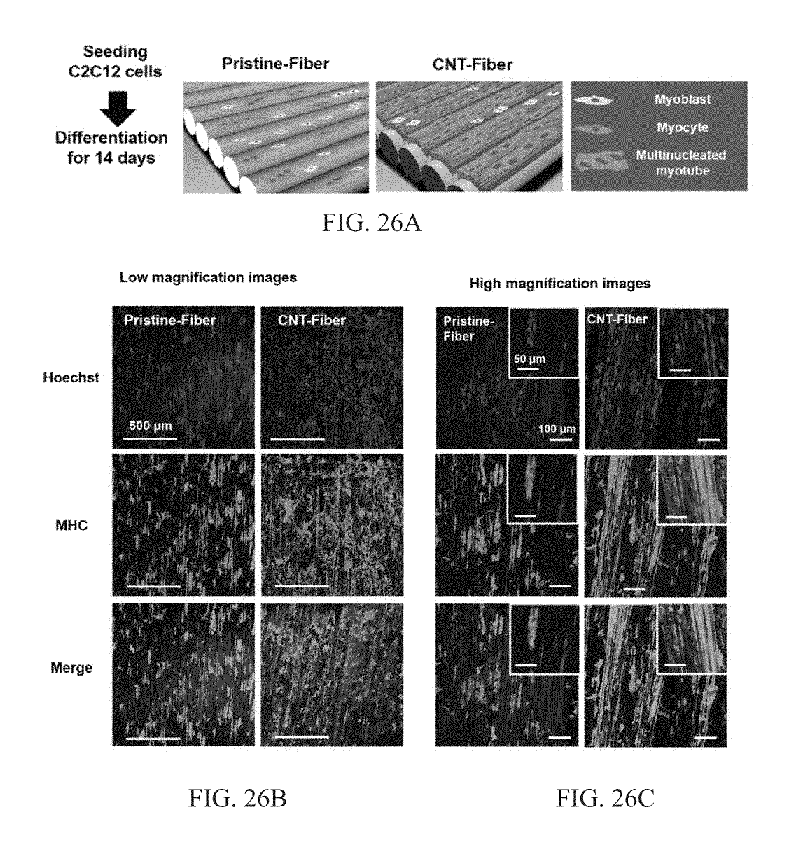

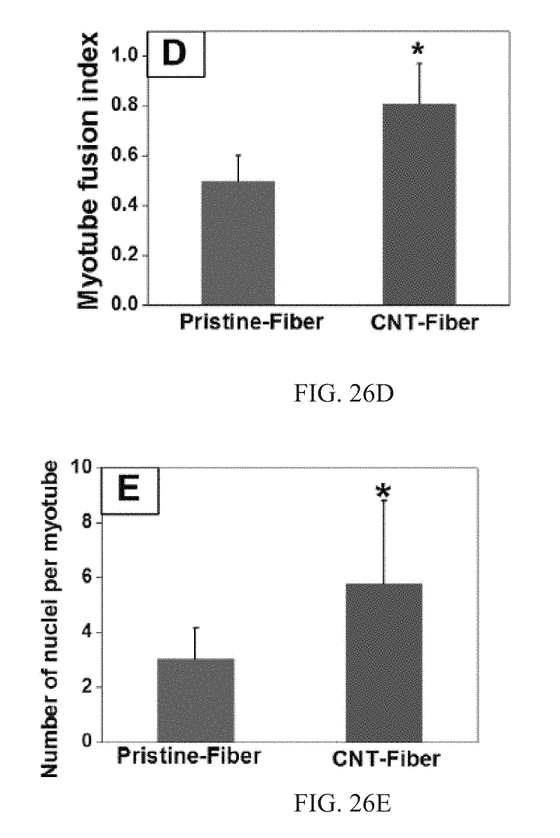

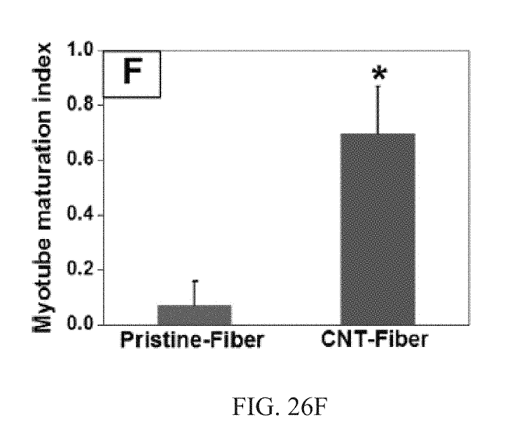

FIG. 26 illustrates effects of fiber alignment on myoblast differentiation into multinucleated myotubes for embodiments of the innovation. (A) A schematic showing C2C12 myoblast cell differentiation seeded on Pristine-Fibers and Carbon Nanotube-Fibers over a period of 14 days; (B) Low magnification (10×) confocal microscopy images showed differentiation of C2C12 cells into myocytes as indicated by myosin heavy chain (MHC, green) staining. Nuclei were stained with Hoechst (blue). In Carbon Nanotube-Fiber, almost all the cells stained for MHC. Interestingly, Carbon Nanotube-Fibers showed continuous multinucleated cellular structures spread across the entire image area. Scale bar represents 500 μm; (C) High magnification (20×) confocal microscopy images showing formation of continuous multinucleated myotubes indicated by MHC and nuclear staining. Very high magnification (80×) images (insets) revealed formation of continuous multinucleated myotubes similar to muscle fiber bundles. Scale bar represents 100 μm and 50 μm for main and the inset images respectively. (D) Myotube fusion index (cells containing 2 or more nuclei/total number of cells per image); (E) Number of nuclei per myotube; (F) Myotube maturation index (ratio of myotubes containing 5 or more nuclei to total number of myotubes). *shows significant difference at p<0.05 compared to Pristine-Fibers; statistical analysis was done using student t-test.

In another embodiment, the integrated influence of fiber alignment and nano-roughness of pristine-fiber and carbon nanotube-fiber mats on differentiation of C2C12 myoblast cells over an appropriate time frame, for example, 14 days may be shown (Schematic in FIG. 26A). 14 days may be selected in pristine-fiber and carbon nanotube-fiber scaffolds as reflecting the time frame of most of the confluent cellular structures formed during 21 days in other example embodiments were detached from the mats preventing any analysis of MHC-positive cells (data not shown). This suggested that aligned fibrous mats might be more efficient in promoting differentiation at earlier time point. Indeed, low magnification confocal microscopy images of cells stained for MHC (green) and nuclei (blue) showed MHC-positive cells in both pristine-fiber and carbon nanotube-fiber mats in contrast to the foam scaffolds.

In the example embodiment, it is noteworthy that while pristine-fiber mats showed MHC-positive multinucleated cells scattered throughout the image area (FIG. 26B, left column), carbon nanotube-fiber mats showed interconnected, multinucleated MHC-positive cells covering entire area of the image, indicating efficient fusion of differentiated myocytes to form myotubes (FIG. 26B, right column). Higher magnification images of both pristine and carbon nanotube-fiber mats displayed presence of aligned myotubes parallel to the fiber direction (FIG. 26C). Interestingly, unlike Pristine-Fibers, Carbon Nanotube-Fibers promoted formation of continuous myotube bundles (inset of FIG. 26C), which are suggested to be units of functional muscle tissue.

Image analysis of the example embodiment further revealed significantly higher myotube fusion index in Carbon Nanotube-Fibers compared to Pristine-Fibers (FIG. 26D). Similarly, number of nuclei per myotube (FIG. 26E) and myotube maturation index (myotubes containing more than 5 nuclei) (FIG. 26F) were found to be significantly higher in carbon nanotube-fiber mats compared to pristine-fiber mats. Immunofluorescence images of MHC-stained cells also showed striations perpendicular to the direction of myotube length in Carbon Nanotube-Fiber (FIG. 31, asterisks), whereas such striations were absent in pristine-fiber scaffolds. Presence of such striations indicates maturity of myotubes in Carbon Nanotube-Fiber. This observation correlates well with the 4-5 fold increase in myotube maturation index on Carbon Nanotube-Fiber compared to pristine-fiber scaffolds (FIG. 26F).

In yet another embodiment, the innovation's aspect of improved myocyte fusion was evident in pristine-fiber mats that consist of aligned fibers of about 10 micron diameter (as opposed to fibers alone). Nano-functionalization with carbon nanotube further facilitated myocyte fusion leading to multinucleated mature myotubes. Taken together, nano-functionalized Carbon Nanotube-Foams promoted myoblast differentiation into MHC-positive myocytes (FIG. 23E); however, it was not sufficient to stimulate fusion of differentiated myocytes into multinucleated myotubes (FIG. 23D(D6), 23D(D9)). On the other hand, exhibiting aspects of the innovation, adding structural cue of aligned fibrous architecture of Pristine-Fiber alone promoted myocyte differentiation as well as their fusion into multinucleated myotubes to some extent (FIGS. 26C and 26D). More advantageously, nano-functionalization interfaced with microscale aligned fibrous architecture in Carbon Nanotube-Fiber significantly, which enhanced myocyte fusion into multinucleated and mature myotubes, highlighting synergy between surface nano-topography and aligned fibrous architecture (FIG. 26C-26F).

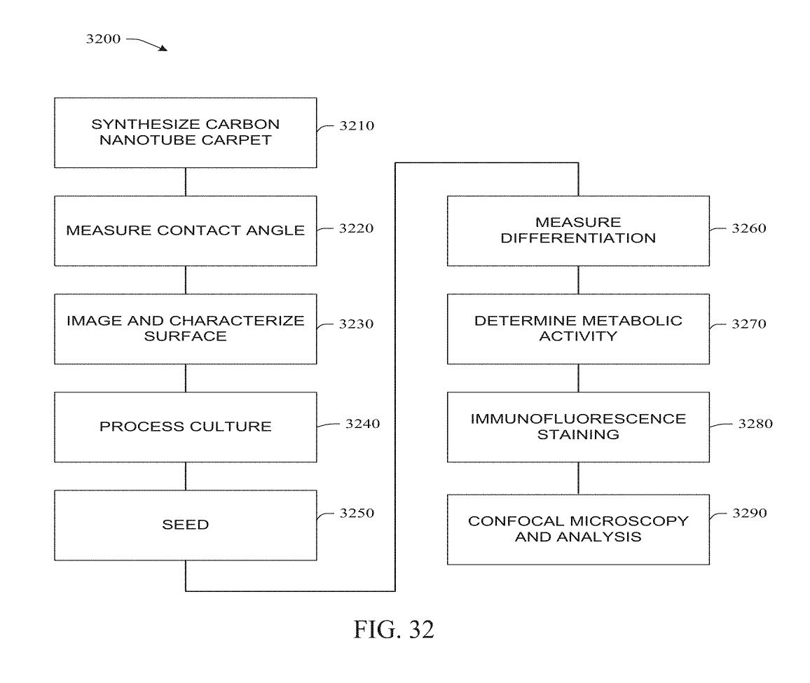

Now turning to FIG. 32, an example embodiment method of nano-functionalization of carbon foams and fibers and analysis can be undertaken will be discussed in steps through a process reflecting aspects of the innovation. The starting substrate can be in the form of foam, woven fiber, unwoven paper or hybrid material. Key in selection is that it offers the suitable structural and mechanical property needed for the application (e.g. flexible fibers for muscles and neurons versus rigid foams for bone, varying porosities for different types of bone, muscle or neural tissues etc.). These substrates can be selected from a large variety of commercially available fabric and foam structures.

At step 3210, carbon nanotube carpets may be synthesized using a two-step process previously developed by Mukhopadhyay group. The first step may involve deposition of silicon oxide activation/buffer layer via room temperature Microwave Plasma Enhanced Chemical Vapor Deposition reactor. The hexamethyldisiloxane [HMDSO, Sigma Aldrich, 99.5%] can be used as a precursor in conjunction with 99.9% pure oxygen. This may be followed by chemical vapor deposition of carbon nanotube using xylene and ferrocene [Alfa Aesar, 99%] as a carbon and catalyst source, respectively. Silica (SiOx) coating can be applied using methods published previously and known to the art. Briefly, acid catalyzed sol-gel process may be utilized in order to produce the silica coating on the carbon nanotube-grafted hierarchical substrate using Tetraethyl orthosilicate (TEOS, Sigma-Aldrich; reagent grade, 98%) as a precursor. Plain weave carbon fiber mats may be obtained from Hexcel (ACGP206-P) and can be functionalized with carbon nanotubes as described above for the foams.

Measurement of contact angle may occur at step 3220. The contact angles of pristine and functionalized foams and fibers can be measured using a dynamic contact angle goniometer set-up such as may be found in the Mukhopadhyay laboratory. Details of this well-established experimental set-up are known in the art. A drop of de-ionized water can be added on the samples using a 21G needle and the image may be captured after 10 seconds. The images may then be processed and contact angle measured using software such as, for example, SolidWorks.

Step 3230 Image and Characterize Surface may use, for example, Scanning electron microscopy (SEM) may follow in order to image and characterize the surface of as-prepared and cell-seeded Foam and Fiber scaffolds. This step may be carried out using, for example, equipment such as SEM (JEOL 9335 Field Emission SEM). Cell-seeded scaffolds may be fixed following an appropriate time schedule, for example, 21 days and 14 days of culture for Foams and Fibers, respectively. SEM may be performed following an appropriate air drying time, for example, 24 hours. Scaffolds may be sputter-coated with 5 nm of gold-palladium using appropriate equipment, for example, a Cressington 108 auto sputter coater. Images may be obtained using accelerated voltage of 3 kV and a working distance of 8 mm. Once these images are obtained, an elemental analysis using energy dispersive spectroscopy (EDS) may be performed. Elemental composition may be determined using energy dispersive spectroscopy (EDS) at an appropriate operating voltage, for example 7 kV. When the solid to be analyzed is exposed to a beam of electrons in the Scanning Electron Microscope, the atoms in the sample emit secondary, backscattered X-rays, whose energies are characteristic of the atomic element. By analyzing the peaks of X-ray energies using EDS detector, elements present in the sample may be identified.

At step 3240, a culture of model cell line (for example, mouse myoblast C2C12, human fetal osteoblast (hFOB 1.19, ATCC, CRL) or human neuroblastoma SH-SYSY cell line) may be processed to show certain aspects of the innovation.

At step 3250, cell seeding on foams and fibers may take place. In an example embodiment, cylindrical foams of height 0.5 cm and diameter 0.5 cm may be sterilized for 30 min in 70% isopropanol under UV. The foam scaffolds may be seeded with a seeding density of 1×10⁶ cells/scaffold. Fiber mats of size 1 cm×1 cm may be seeded with C2C12 cells at a seeding density of 90,000 cells/mat (normalized based on the volume of mats). This portion of the example method may be carried out over an appropriate period of time, for example a period of 14 days.

For measurement of the aspect of differentiation, as in step 3260, the scaffolds may be cultured in growth media until day 3 followed by additional 18 days in differentiation media (for example, DMEM supplemented with 10% heat inactivated horse serum, HyClone™).

At step 3270 the metabolic activity of cells can be determined. For measurement of the aspect of metabolic activity, cells may be seeded and cultured for 14 days in growth medium. The metabolic activity of cells seeded on Foams or Fibers (n=3) can be measured using for example the Alamar Blue® assay (Thermo Scientific, USA). The assay can be performed over an appropriate period of time, for example, a period of 14 days. Alamar Blue® solution (10% v/v) can be prepared in complete growth media and 1 mL of this solution can be added to each well containing cell-seeded scaffold and incubated for 4 hours at 37° C. After this step, 100 μL of the solution from each well can be transferred to 96-well plate and the fluorescence intensity may be measured at an appropriate excitation/emission wavelength, for example, 530/590 nm, using a microplate reader (such as a Synergy HT, BioTek instrument). The wells containing only Alamar Blue® solution in media may be used for background fluorescence correction. Pristine-Foams and Pristine-Fibers may be used as control to compare the proliferation on functionalized Foams and Fibers.

At step 3280, immunofluorescence staining may be performed. Cell-seeded Foams or Fibers can be fixed, for example, with 4% paraformaldehyde for 30 min and washed 3 times with DPBS followed by the blocking/permeabilization with, for example, DPBS containing 0.1% Triton X-100 and 5% BSA for an appropriate time, for example, for 1 hour at room temperature. The scaffolds may then be incubated with the primary antibody against myosin heavy chain (MHC), MF-20 (1:50, DSHB, Iowa) overnight at 4° C. and washed with DPBS three times. The scaffolds may then be stained with secondary antibody (such as Alexa Fluor 488-conjugated goat anti-mouse IgG, 1:1000, available at Santa Cruz Biotechnology Inc., USA) for an appropriate amount of time, for example for 1 hour at room temperature followed by three times washing with DPBS. The cell nuclei may be stained with Hoechst 33342 (Thermo Fisher, USA).

At step 3290, confocal microscopy and analysis may be performed. Confocal images can be obtained using an inverted confocal laser scanning microscope (such as Olympus Fluoview 1000 or the like). Lasers of appropriate wavelength, for example, 488-, 559-, and 633-nm wavelength may be used. In the example embodiment, objective lenses of 20× and 40× were used to acquire the z-stack images with 5 μm thickness of each z slice. Data may be presented as maximum intensity projection of the z-stack. Once so presented, software such as NIH ImageJ software may be used for quantification of the confocal images. Percentage MHC-positive cells may be calculated by taking a ratio of MHC-positive cells to the total number of cells in at least 3 images per scaffold type. In the example embodiment, at least 120 total nuclei were considered per scaffold type. Myotube fusion index may be calculated by taking a ratio of number of fused cells containing 2 or more nuclei to total number of nuclei per image for the Fiber scaffolds. In the example embodiment, the number of nuclei per myotube was measured manually. Myotube maturation index may be measured from the immunofluorescence images by calculating ratio of myotubes with 5 or more nuclei to the total number of myotubes. The graphs may be plotted using appropriate tools, such as for example GraphPad Prism 6 and Origin Pro 2015. Further, the statistical analysis of the data can be represented as mean±standard deviation (n=4-5). The statistical significance between the groups may be analyzed using one-way or two-way ANOVA for multiple comparisons followed by Tukey post-hoc analysis (available with for example, GraphPad Prism 6). In the example embodiment, p values less than 0.05 were considered statistically significant.

### EXAMPLES

### Example 1: Carbon Nanotubes on Silica Coated Graphite

Base substrates used were flat graphite as well as microcellular foams of graphitic carbon. These were modified by coating with a silica nano-layer using microwave plasma deposition (MPD) of hexa-methyl-disiloxane (HMDSO) in O₂. Deposition of the oxide coating intermediate layer was accomplished in stages: (stage 1) O₂ (99.99%) gas introduced into the microwave plasma chamber to clean the surface; (stage 2) O₂ and HMDSO introduced at microwave power of 250 W; and (stage 3) O₂ carrier gas introduced to stabilize the oxide.

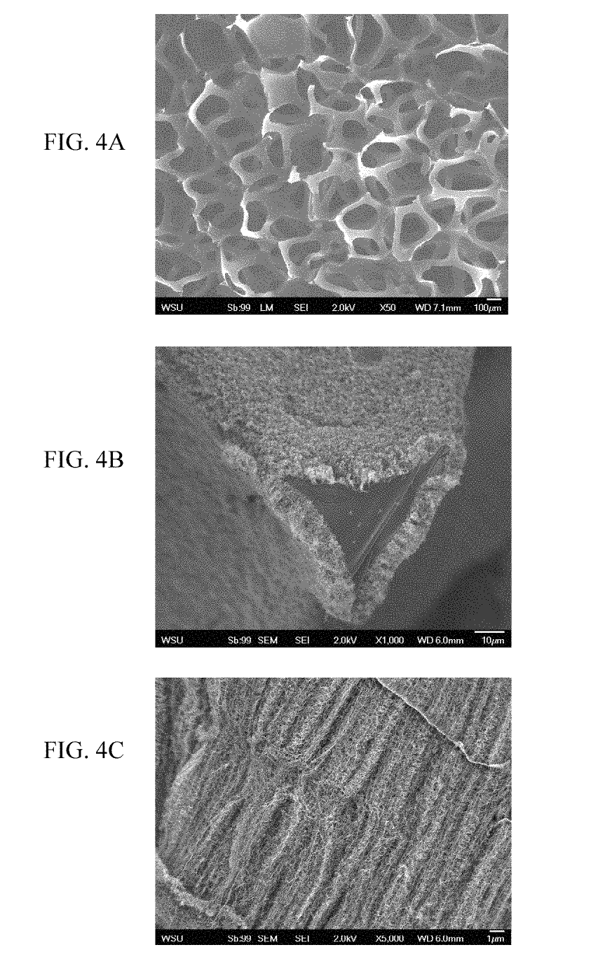

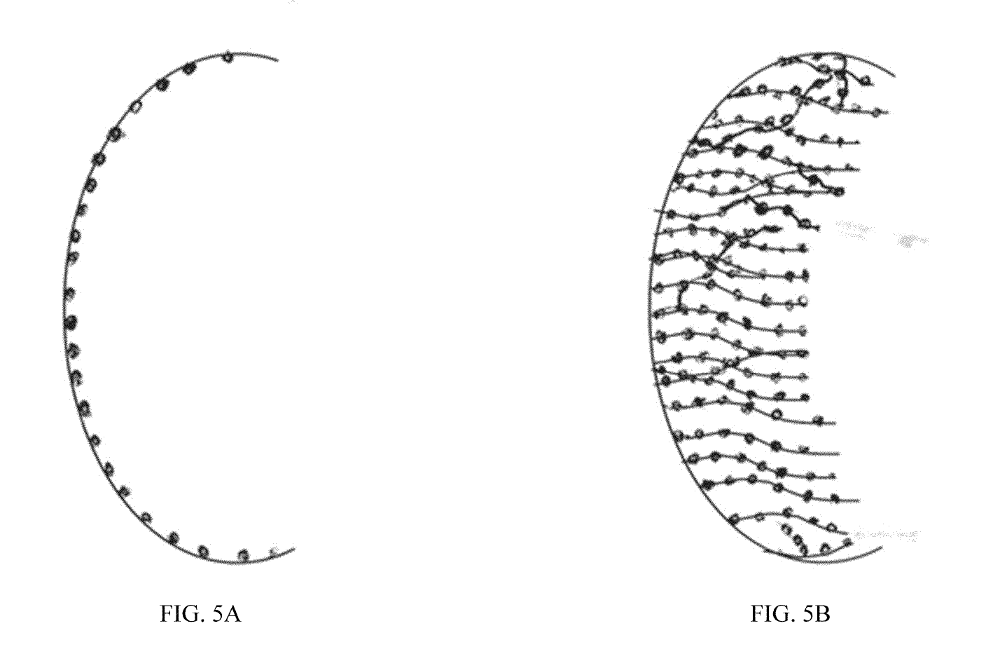

After silica layer deposition, samples were transferred to a CVD reactor for carbon nanotube (CNT) deposition. The CVD process parameters were optimized for purest nanotube growth. Optimization conditions depended upon the exact geometry of the substrate to be modified. The first stage of the CVD is used to heat the ferrocene/xylene solution prior to injection. The second stage of the CVD furnace is heated to the growth temperature for carbon nanotube fabrication (700-800° C.). Once samples were positioned in the reactor, a measured solution of ferrocene (C₁₀H₁₀Fe) dissolved in xylene (C₆H₄C₂H₆) was injected into a flowing gas mixture of argon and hydrogen. The substrates were heated to the growth temperature and subjected to deposition for specific times. After the allotted time, samples were allowed to cool in argon before removal from the CVD chamber. The deposition parameters for optimum nanotube morphology depended upon the initial geometry of the surface to be coated. FIG. 3 demonstrates a dense growth of carbon nanotubes is obtained on carbon foam as in this example using the surface modification technique of plasma pretreatment in combination with subsequent CVD deposition. The pretreatment level can be used to control the density of nanotubes grown on the surface, and the CVD process parameters can be adjusted for control of nanotube length and quality. FIG. 4 shows the growth of nanotubes using this method on reticulate carbon foam. FIG. 4A is a low magnification (50×) image of the foam as purchased. FIG. 4B (1,000×) shows that each strut can be covered with a dense carpet of nanotubes and FIG. 4C (5,000×) shows that the nanotube ‘forest’ can be very dense for long enough CVD times.

Semi-empirical predictions obtained by combining analytical modeling and micro-structural data indicate that it would be easy to obtain at least 100 times or more increase in surface area using the concepts of the subject innovation.

Direct specific surface area (SSA) measurements using the Brunauer-Emmett-Teller (BET) technique with nitrogen has been performed on cellular foam substrate material. It is seen that in commercial porous foams having a starting surface area of about 0.017 m²/g, the attachment of nanotubes results in a surface area of about 3 m²/g, implying an increase of well over 100-fold. Also significant, this occurs with negligible increase in volume and a weight gain of only about 2.5%. These advantages are anticipated to be significantly boosted for even longer grown nanotubes.

Thermal dissipation behavior of such structures into phase change materials has been tested and seen to be promising. The nanotube attachments are seen to increase the mechanical interlocking and prevent delamination between these solids and any matrix material, hence making them suitable for use in advanced composites.

These structures are also seen to offer accelerated growth of biological cells due to the scaffolding action of nanotubes on the surface. CNT-grafted foams show more prolific growth of healthy cells, which may result in faster bio-integration and healing of implants.

In addition to these advantages, at least one advantageous benefit of these hierarchical structures may come from the fact that the increased surface area can now harbor larger number of functional nanoparticles on its surface. FIG. 5 shows the schematic of the surface profile, indicating why a pore wall with attached nanotubes can support larger number of functional nanoparticles for applications such as catalysis, sensing, hydrogen storage, or antibacterial activity.

### Example 2: CNT and PdNP on Graphitic Supports

Base structures tested were microcellular and reticulated carbon foam having uneven and irregular geometries as well as flat graphite substrates. Analytical reagents used, without further purification, were as follows: hexamethyl-di-siloxane (HMDSO), xylene, ferrocene, tetraamine palladium (II) nitrate solution (TAPN), methanol and concentrated nitric acid (HNO₃, 70%), and distilled water.

The as-received carbon foam substrate was modified, or pretreated, prior to attaching palladium nanoparticles by (1) nitric acid etching and (2) plasma assisted silica coating. Nitric acid etching was performed by immersing the carbon foam in 16M HNO₃ for a few minutes followed by sonication with distilled water to ensure complete removal of acid. Silica nanocoating was deposited onto the substrates in a microwave plasma reactor using HMDSO. The coating time was 15 minutes.

As an alternative to the above pre-treatments, some studies were also done on foam samples grafted with carbon nanotubes using the process discussed in Example 1. On all these samples, palladium nanoparticles (PdNP) were fabricated by liquid-phase synthesis technique followed by thermal reduction process. Tetraamine palladium nitrate (TAPN) was used as the metal precursor solution. Supports, cleaned with methanol and distilled water prior to infiltration, were immersed in the aqueous Pd precursor solution of tetraamine palladium (II) nitrate (TAPN) for specific period of time. The molar concentration used in this study was 62.5 mM TAPN and the supports were impregnated in the TAPN solution for 30 mins. The solid supports were recovered from the TAPN solution and the excess non-interacting solution on the sample was washed by briefly dipping it in methanol. The samples were placed on a ceramic boat and immediately transferred to the furnace for heat treatment processing. Heat treatment may include several individual steps, or using a combination of steps such as: drying, calcining, and reducing. In one example, the impregnated samples were dried at 100° C. for 12 hrs in the ambient atmosphere to eliminate water from the samples. Calcination was then carried out in either oxygen-rich ambient atmosphere (air) or oxygen-deficient inert atmosphere (Ar). The thermal profile in this step was controlled with a ramp rate of 10° C./min and held at 450° C. for 2 hrs. This process of controlled heating was adapted to avoid sintering. The final step involved thermal gas-phase reduction of Pd oxides to metallic Pd. In this example, the temperature was increased to 500° C. and held for 2 hrs using hydrogen gas (25 cc/min) as a reducing agent in an inert atmosphere of Ar (500 cc/min), Ar/H₂:20/1. The furnace was then allowed to cool down to room temperature in the reduced flow of Ar and H₂. Surface morphology of metallic nanoparticles and hierarchical structures were observed using JEOL 7401F Field Emission Scanning Electron Microscopy (FESEM). Statistical analysis was carried out on SEM micrographs using Scandium© SEM imaging software for JEOL 7401F FESEM.

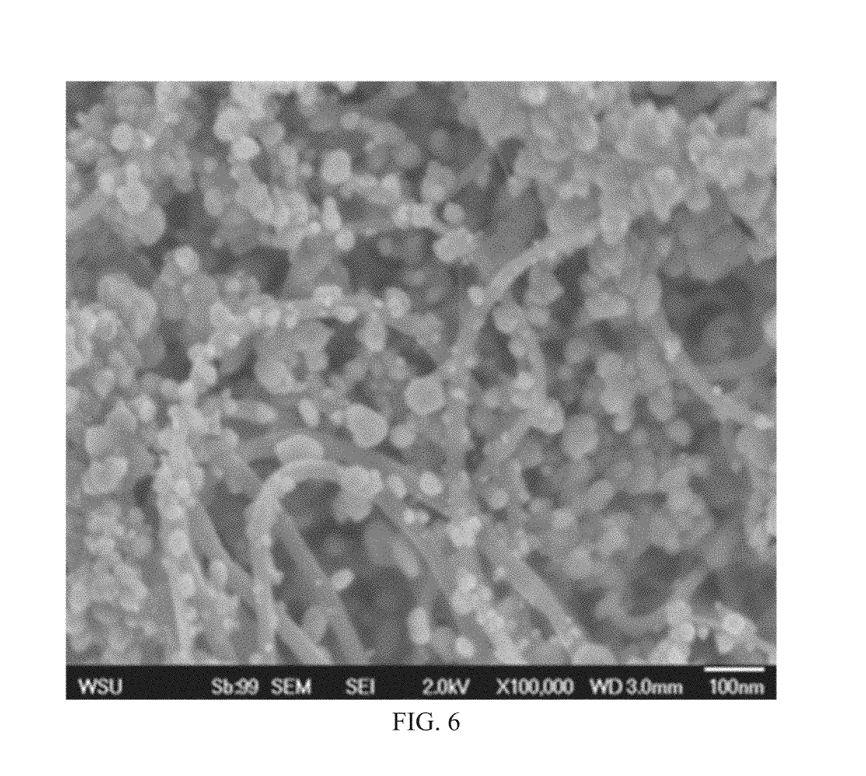

FIG. 6 demonstrates nanotubes attached to cellular carbon foam and subsequently functionalized with Pd nanoparticles.

### Example 3: CNT and AgNP on Graphitic Supports

Several supports, or substrates, were tested for attachment of silver nanoparticles. They include (i) cellular carbon foam (ii) flat HOPG graphite (iii) carbon fibers and (iv) paper foils made of nanofibers, grapheme, and nanotubes.

Some of the samples were used “as-is,” while others were exposed to CVD to attach nanotubes as discussed in Example 1.

These structures were pre-wetted with methanol followed by dipping it in 0.24 M AgNO₃ solution for approximately for 1 hr. Samples were then placed on hot plate at 100° C. for 30 mins to remove moisture/water. These were finally reduced to metallic silver nanoparticles using the following process: DMSO has taken in a reduction beaker and a magnetic stirrer was placed at the bottom of the beaker. The sample was placed on a shelf above the stirrer. The reduction beaker was placed on a hot plate and heated to a temperature of from about 60° C. to about 80° C. followed by addition of 5 mg tri-sodium citrate. Continuous stirring helps rapid dissolution of citrate in DMSO. Silver nitrate coated sample was then placed on the sample shelf. After the desired time, samples were taken and washed off with distilled water and left to dry.

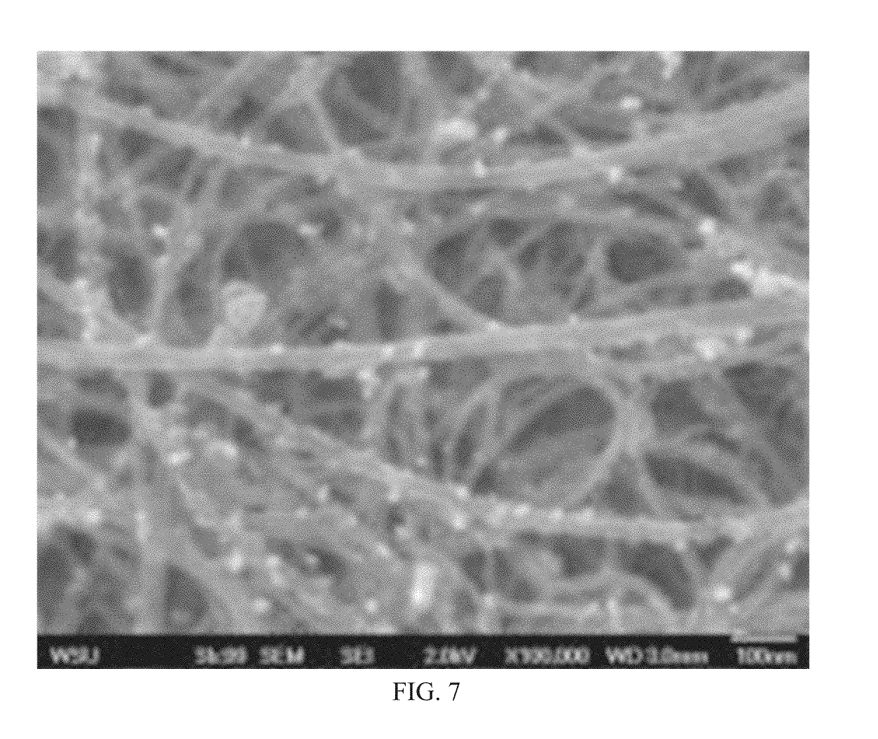

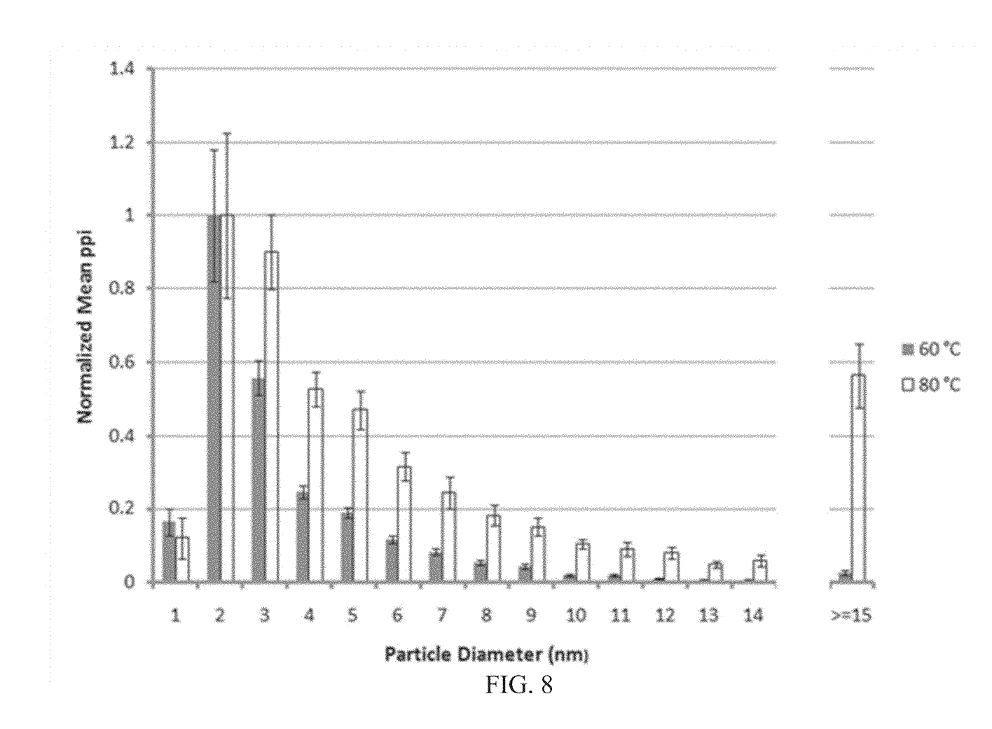

The particles were characterized using Field Emission SEM and detailed measurements of particle size distribution were made. FIG. 7 demonstrates nanotubes attached to carbon substrates, which are subsequently functionalized with silver nanoparticles (AgNP). FIG. 8 is an example of the particle distribution obtained for Ag nanoparticles obtained at two reduction temperatures. The distribution may also be altered to some extent with initial salt concentration.

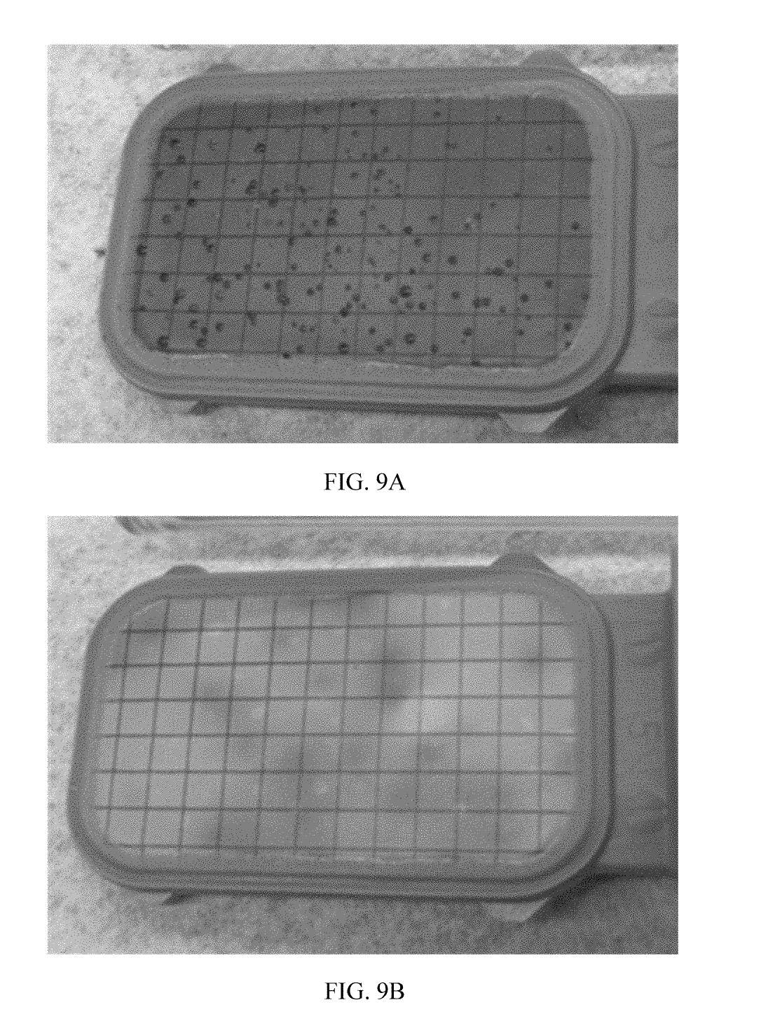

The anti-bacterial activity of these structures was measured with the idea of incorporating them into fluid purification systems. FIG. 9 shows the influence these materials have on lake water. FIG. 9A is the control, which shows a large number of bacterial colonies (dark spots) formed by incubation of untreated lake water. FIG. 9Bb shows results from identical testing performed on the lake water, after it was rotated in a 1-liter bottle containing 4 mm specimen of AgNP-decorated CNT-Foam support. The bacterial colonies are reduced drastically, or are non-existent.

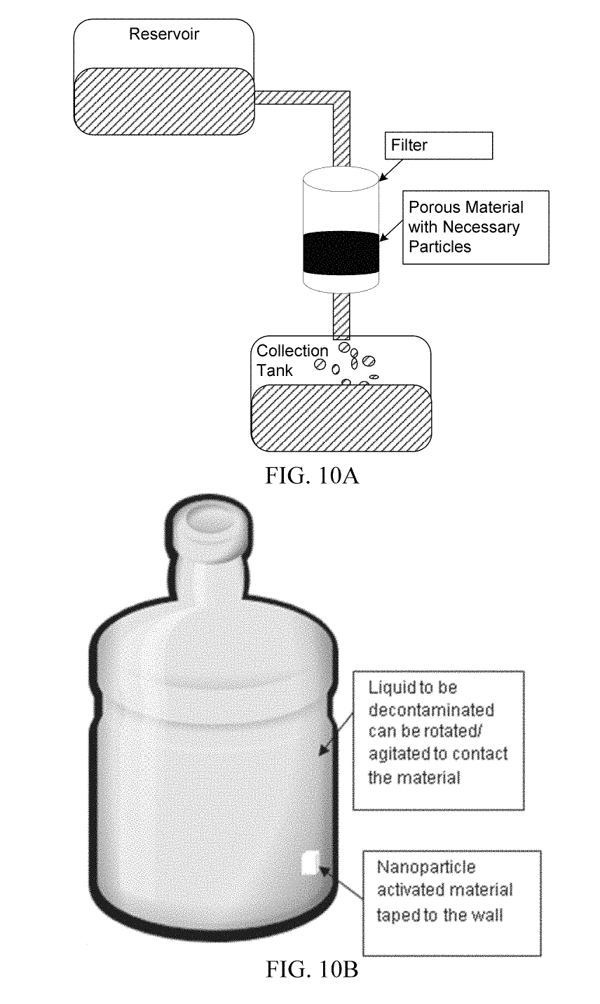

It is anticipated that the structures in this invention can be utilized in several different configurations for water purification, two of which have been tested successfully so far: flowing the contaminated water through the porous foam (as a filter shown in FIG. 10A), and rotation of Ag-NP decorated foam in 4000 times its volume of contaminated water (FIG. 10B).

### Example 4: Hydrophilic Coatings to Enhance Functionality in Aqueous Environments

Carbon nanotube (CNT) grafted hierarchical surfaces offer high specific surface areas, a defining parameter for many applications involving solid-environment interactions. However, CNT grafted hierarchical surfaces tend to be super-hydrophobic due to their inherent physical and chemical characteristics. Control over surface wettability can play an important role in maximizing the surface-fluid interactions. In aspects of the subject innovation, two different types of surface modification techniques can be employed: techniques that can permanently make the surface hydrophilic, and techniques that can make the surface reversibly hydrophilic. Example techniques include dry etching via oxygen plasma to make the surface reversibly hydrophilic, and silica coatings via a liquid based sol-gel method to make the surface permanently hydrophilic. While plasma etching and heating could impart switchable wettability, silica coatings caused permanent wettability. Structure, morphology, composition and chemistry of these materials were investigated, and related to surface wettability and water flow. It was seen that plasma treatments did not alter the surface morphology in any way, but did change the chemical composition, which can be related to wettability. Sol-gel treatment coated the nanotubes with an amorphous layer of pure SiO₂. Water contact angle (CA) measurements discussed herein demonstrate that the plasma coating reduces contact angle, which can be subsequently restored by heat treatments. Silica coatings, on the other hand, impart permanent decrease in contact angle. Comparison of water flow through porous foams coated with CNT arrays and subject to similar treatments showed that hydrophilic coatings can significantly improve the flow along these surfaces due to increased wettability and capillary action. These results have important implications on many surface related applications of these materials such as catalytic supports, antimicrobial filters, microfluidic devices and environmental remediation.

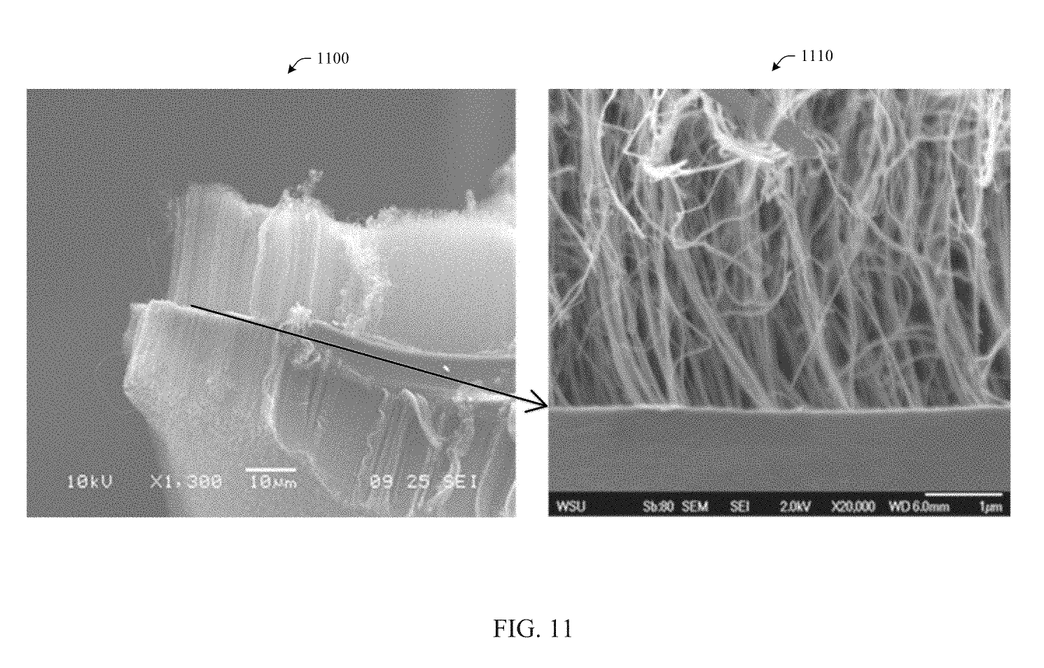

FIG. 11 illustrates ultrahigh surface area hierarchical substrates at 1100 and 1110, in accordance with aspects of the subject innovation, which can be very useful as supports for nanocatalysts, sensors and advanced composites. Many of the potential applications, such as water purification, catalysis etc., will involve flow and interaction of fluids through the nanotube carpet attached to their surfaces. Controlling the flow of aqueous fluids through this region can therefore be very useful.

In general, surface wettability of commonly known carbon-based structures such as graphite, carbon fibers, and isolated carbon nanotubes can be controlled by functionalization with oxygen-containing radicals via liquid-phase or gas-phase oxidation. Molecular groups such as hydroxyl, carbonyl, ester, and nitro groups can be introduced on the surface by liquid-based treatments with strong acids, peroxides, or gas-phase thermal or plasma treatments involving oxygen, Carbon dioxide, UV/Ozone radiation, air/water vapor and similar approaches. Multiple variations of one or more of these methods have been reported for conventional carbon materials by several investigators. In various embodiments, the subject innovation includes one or more techniques suitable for controlling the wettability of hierarchical multi-scale carbon materials such as those discussed herein and shown in FIG. 11.

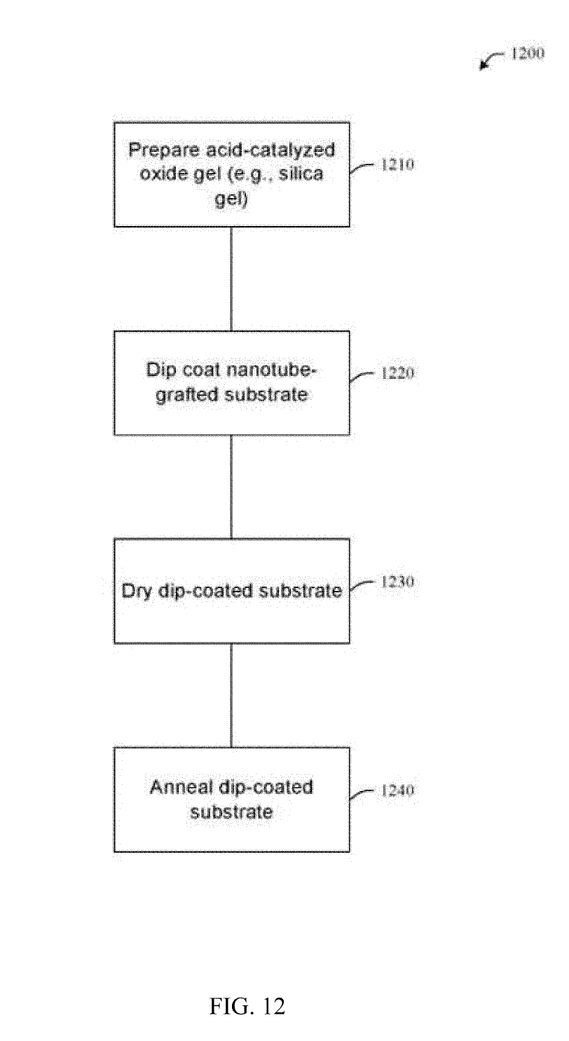

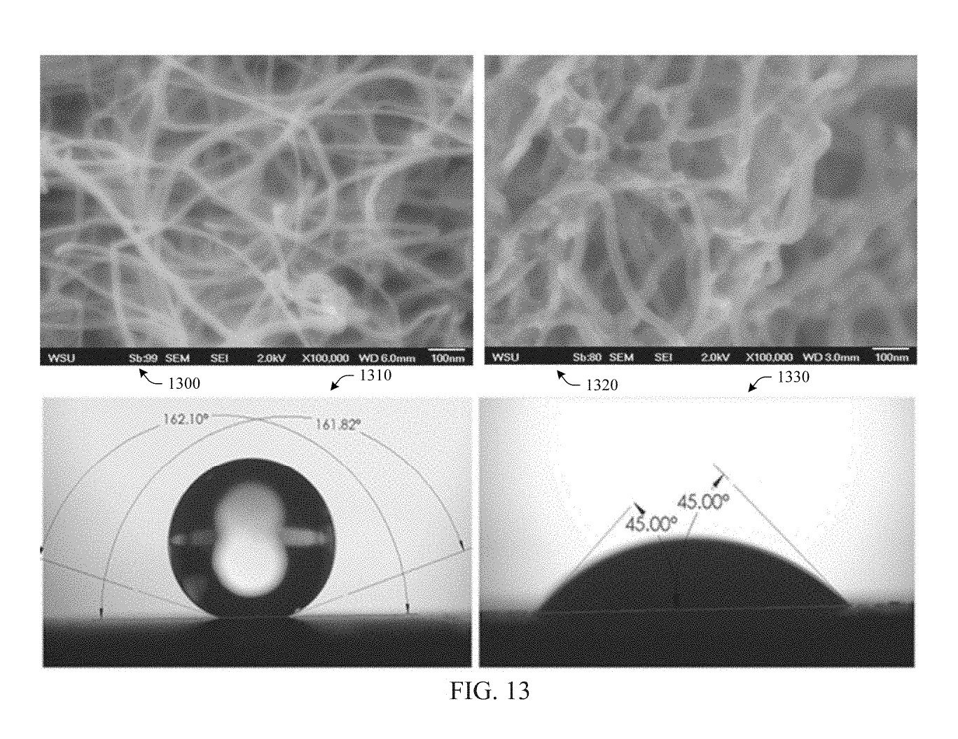

In permanently hydrophilic embodiments, this modification can include uniform coating of individual nanotubes in the carpet with a 1-3 nm layer of a polar oxide such as silica. This can be achieved by careful modification and optimization of a sol-gel process applied to traditional larger materials. FIG. 12 illustrates a method 1200 of applying an oxide coating to a nanotube-grafted substrate in accordance with aspects of the subject innovation. Method 1200 can involve preparing an acid catalyzed oxide gel (e.g., silica gel, etc.) at 1210, dip coating the nanotube-grafted substrate at 1220, drying (e.g., air drying, etc.) the substrate at 1230, and annealing (e.g., in inert atmosphere, etc.) the coated substrate at 1240. One difference between this method and a traditional sol-gel approach is the possibility of carefully optimizing the viscosity and permeability of the starting precursor solution (silicon containing molecule, alcohol and water with or without other solvents) that can enable precursor permeability and contact into the given CNT carpet having specific CNT densities and lengths. In this way, individual nanotubes in the carpet can be uniformly coated with the precursor before it starts gelling, so that clumps and flakes of oxide are avoided. Although only a few windows of precursor compositions are discussed herein, a spreadsheet recipe can be developed for different grades of application specific hierarchical substrates. FIG. 13 shows images of a CNT hierarchical surface before coating at 1300 and after silica coating by the technique of method 1200 at 1310, and their respective responses to a drop of water at 1320 (before treatment) and 1330 (after treatment), showing contact angles for water before and after treatment. It can be seen that individual nanotubes are coated with the silica at 1310, and that the water drop spreads more easily on the treated surface at 1330. Such treatments have been tested on porous carbon foams coated with nanotubes, and it has been seen that the flow of water, as well as the interaction of the water with nanotube walls (for the needed catalytic or antibacterial application), was significantly increased by such a treatment.

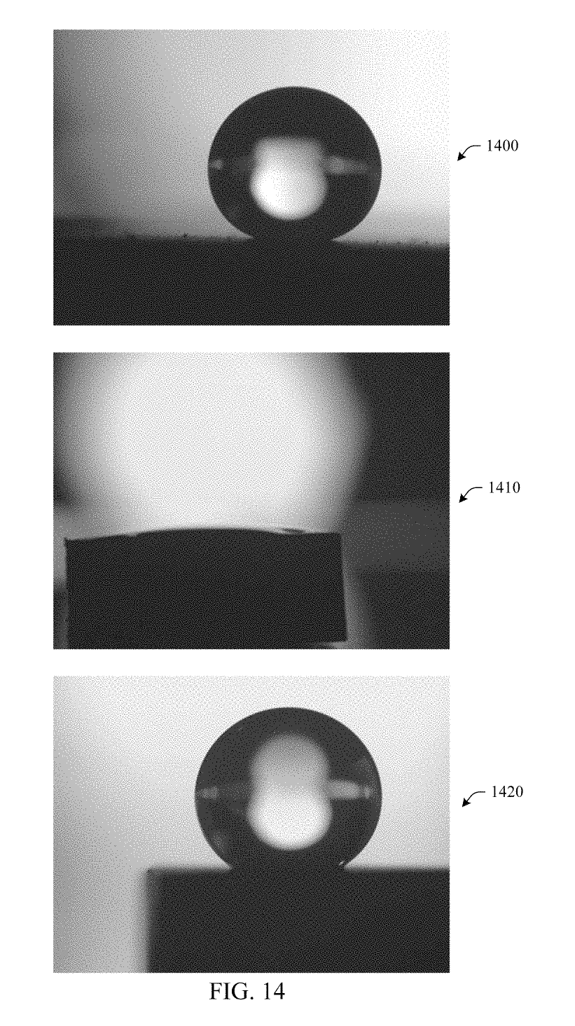

In plasma coating embodiments, plasma coating can be accomplished by treating the material in a low-medium power microwave plasma of oxygen gas for very short times (2-8 sec). An advantage of this approach is that this treatment is reversible, and hydrophobicity can be restored by simply heating the material in air at 100° C. This makes this low cost approach very suitable for applications where temporary wettability is desired. FIG. 14 shows images of a water droplet on an initial CNT forest at 1400, after plasma treatment at 1410, and after reheating in air at 1420.

In various embodiments, the subject innovation includes methods, compositions, and articles related to strategic control of the wettability of water and other polar fluids on ultra-high surface area hierarchical substrates. Control of wettability can be used to further enhance the properties and applicability of ultra-high surface area substrates. A well-aligned carpet of pure carbon nanotubes (CNT) on a porous substrate tends to be inherently hydrophobic in nature (it repels water away from the nanotube carpet). This hydrophobic nature can be attributed to both the surface chemistry of carbon nanotubes and to the hierarchical morphology of the CNT carpet. This hydrophobic nature may be useful for some applications, but not for others, which may need deeper permeation of water through the CNT forests. The latter applications will benefit if the CNT forests can be rendered hydrophilic. In various aspects, the subject innovation can employ either of two types of techniques for making the surface hydrophilic by attachment of oxygen containing species on the walls of the CNT, a first type of technique to make the surface permanently hydrophilic, or a second type of technique that can make the surface reversibly hydrophilic. In an example of the first type of technique, a liquid phase method of coating the CNT forest with a nanoscale oxide layer is described herein, which can make the surface permanently super-hydrophilic. In an example of the second type of technique, a dry plasma etch technique can be employed that can make the surface reversibly hydrophilic, wherein the hydrophilic behavior can be reversed by a simple heat treatment and reapplied when needed. This second type of technique can allow cycling of the material between hydrophilic and hydrophobic states. In additional embodiments, surface treatments can be applied to vary the interactions of nanostructures with their environment in other manners, such as to provide coatings with effects ranging from catalytic, photocatalytic, antimicrobial, petroleum cracking, environmental cleanup, alterations in oleophobicity, etc.

High porosity solid structures with strongly attached Carbon nanotube (CNT) arrays can offer many of the inherent nanoscale advantages while minimizing the environmental risks by anchoring them on robust, easy to handle, solids. Various estimates have shown that the SSA of these types of CNT-modified structures is easily several orders of magnitude higher than the starting porous solids. These multiscale morphologies have many applications in reinforcement for structural and thermal management composites, liquid purifications devices, tissue scaffolding and sensing. In addition, these materials offer excellent support for functional nanomaterials such as sensors, catalysts and disinfection devices.

According to wetting models for rough surfaces, water contact angle (CA) of inherently hydrophobic surfaces can be increased by “roughness factor” which makes CNT grafted surfaces super hydrophobic (≥1600). In various embodiments of the subject innovation, techniques can be employed that can improve the surface wettability of CNT grafted surfaces via dry and wet surface treatments.

Wettability is governed by both surface morphology and chemical composition. Surface chemistry of the CNT can be controlled through covalent functionalization by grafting oxygen-containing functional groups via liquid-phase or gas-phase “oxidative” treatments. In liquid phase methods, CNT can be treated with etchants such as nitric acid, sulfuric acid, mixtures of both and/or “piranha” (sulfuric acid-hydrogen peroxide). In gas phase oxidation, CNT can be treated with oxygen (thermal or plasma), carbon dioxide, UV/ozone radiation, or with air/water vapor at high temperatures. While many of these methods increase surface wettability by grafting oxygen-based functional groups, many can destroy the CNT structure itself causing damage. In accordance with certain aspects of the subject innovation, CNT grafted surfaces (or other articles with nanotubes grafted onto a substrate in accordance with aspects of the innovation described herein) can be made reversibly hydrophilic, such as by gently treated with oxygen microwave plasma at room temperature, which can improve the wettability without damage. In this case, the chemical species can be weakly physisorbed, and can be removed by heat treatment. While this approach allows cycling between hydrophobic and hydrophilic behavior, permanent hydrophilicity can also be achieved in accordance with other aspects of the subject innovation. One such technique can involve coating the nanoscale structure with a polar oxide such as silica.

Thin film coatings that completely coat nanotube surfaces without clumping are complicated by their small dimensions. This becomes more challenging when the nanotubes are in the form of arrays on a larger surface, and hence involve a multi-scale solid geometry. In experiments discussed herein, a permanent change in hydrophilicity was induced via a simple solution based sol-gel method for silica coatings, although it is to be understood that examples herein are provided solely for the purposes of illustration, and in various aspects of the subject innovation, similar techniques can be used with other coatings or decorations, which can provide characteristics useable in a range of applications. For example, other oxide or metallic coatings can be employed, such as metal oxides, for example, titanium oxide (e.g., in photocatalytic applications, etc.), mixed metal oxides (e.g., as charge storage devices, etc.), silver (e.g., antibacterial applications, etc.), palladium metal or oxide (e.g., for hydrogen storage, removal of contaminants, petroleum cracking, etc.), etc. The sol gel method can deposit a silica layer on isolated CNT powders. In aspects of the subject innovation, this process can be modified to get uniform or coating of hydrophilic silica (or other uniform or particulate coatings, such as the examples discussed herein, etc.) on the hierarchical solid consisting of CNT-grafted solid substrates (or on substrates with other grafted nanotubes, etc.). These can be equally effective for CNT arrays on planar solids such as graphite as well as on high porosity fabric or foams of any material capable of withstanding temperatures in excess of around 600° C., including but not limited to ceramics, metals, carbon, etc.

Surface morphology and chemical compositions were studied using field emission electron microscope (FESEM) and X-ray photoelectron spectroscopy (XPS) respectively. Crystal was studied using combination of transmission microscope pictures and X-ray diffraction (XRD).

Increased wettability can enable efficient utilization of the extensive surface area offered by the CNT-grafted structures, and also can enhance capillary fluid flow through the CNT carpets. These have important implications for several applications that will benefit from these solids. Experiments discussed herein examined the influence of these surface treatments on wettability and fluid flow. The former was monitored through contact angle measurements, and the latter by pressure drop (ΔP) vs. fluid flux (q) characteristics.

### Wettability Experiments

Two kinds of support structures were used in the experiments: highly oriented pyrolytic graphite (HOPG) and reticulated vitreous carbon foam (RVC) [. The HOPG was a standard flat carbon substrate having well defined geometry, hence used for surface chemistry and wettability studies. The RVC foam, with its interconnected open cell morphology, was used for studying the fluid flow behavior with identical surface modifications.

For the sol-gel process, tetraethyl orthosilicate was used as silica precursor [TEOS; Si(OCH₂CH₃)₄, Si-OEt (Alfaa aesar; 98%)], distilled water (H₂O) for hydrolysis, and absolute ethyl alcohol (EtOH) as common solvent for TEOS and water, and hydrochloric acid (HCl) as acid catalyst for hydrolysis and condensation reactions.

The carbon nanotube grafting was a two-step process; deposition of silicon oxide (SiOₓ) buffer layer, followed by CNT deposition via thermal CVD method using xylene and ferrocene as carbon and catalyst sources respectively. The details of CNT grafting on the selected structures are discussed above. Carbon nanotubes were grafted on both selected support structures.

For surface etching in this experiment, 99.9% pure oxygen gas was used as precursor gas. Carbon nanotube grafted HOPG (CNT-HOPG) surfaces were placed in microwave plasma reactor operating at 115 W. Oxygen gas was fed into vacuum chamber and ionized using microwaves and etched CNT-HOPG surface for 10 seconds.

In reversibly hydrophilic experiments, the hydrophobicity of the CNT-HOPG surface was restored via air annealing at 110° C. for 1 hr.

Silica coating on CNT grafted carbon structure as performed in the example experiments involved multiple steps: acid catalyzed silica gel preparation, silica coating on CNT-carbon via dip coating method. Reaction chemistry of acid catalyzed sol-gel polymerization of TEOS in EtOH and H₂O has been well studied in conventional applications. In this process, silica precursor solution was prepared by mixing TEOS, EtOH, and H₂O in the volumetric ratios of x:1:1 respectively. Keeping the EtOH to H₂O ratio to 1:1, varying concentrations of TEOS to H₂O ratio; 1:2, 1:8, 1:30, and 1:50 were tried to optimize the silica layer thickness on CNT-carbon. The initial mixtures were mixed thoroughly for 1 h followed by adjusting the pH to 3.0 by adding HCl. The solution was further mixed for a 1 h until it became colorless and clear. CNT-carbon structures were soaked in the prepared silica precursor solution for 1 h and followed by air drying for 12 hr. Further drying was carried out on hot plate for 12 h at 100° C. Finally, the samples were annealed at 5000° C. at a rate of 20° C./min in Argon (Ar) atmosphere and kept there for 2 h before cooling them down to room temperature at a rate of 2° C./min. Unlike annealing in air as used in some conventional applications, Ar atmosphere was used to prevent oxidation of CNT and HOPG support.

The morphology, surface chemistry, and crystal structure, of silica-CNT-carbon were characterized using Field emission scanning electron microscope (FESEM), scanning transmission electron microscope (STEM), X-ray photoelectron spectroscopy (XPS) and X-ray Diffraction (XRD) respectively. Microstructure characterization of silica coated CNT-carbon were done using FESEM and STEM. For STEM analysis, silica coated CNT were peeled off from the carbon substrate and loaded on the TEM grids. Crystallographic orientation of synthesized AgNP on carbon structures were studied using X-ray mini diffractometer, MD-10, using a monochromoatized Cu Kα radiation at 25 kV and 0.4 mA. Surface chemistry of processed silica thin film was characterized using XPS system from Kratos (Axis Ultra) using mono-chromatized Al Kα (1486.6 eV) as X-ray source. CASA software was used for spectrum analysis and processing. The carbon is peak at 284.5 eV was taken as a reference position, a well-established value for HOPG, for charge correction.

A simplified in-house built goniometer was used for measuring contact angles (CA). In this set-up, a camera equipped with magnifying lens was mounted on a tripod while the sample is placed on a fixed sample stage. The surfaces were air brushed to remove dust particles and 5 μl of deionized water was gently placed on the sample surface using a micropipette. A box with double window, each on either side of the sample, was placed over the sample. One window was covered with diffuser paper to reduce the light intensity, generate a homogeneous dark background, and minimize reflections at water drop. Finally, the camera height and the distance between camera and the sample were adjusted to precisely determine position of the triple line, the intersection of solid-liquid-air interfaces.

Pictures were loaded in SolidWorks for processing. To measure the CA, a baseline was manually selected by choosing two points (intersecting points of drop profile meeting the flat surface) to define the baseline and three points along the drop profile. Tangents to the profiles were drawn from the meeting point of the profile to the base line and CA was calculated by measuring the angle between tangent and baseline. To improve precision, multiple measurements on multiple drops were obtained using this method and averages and standard deviations reported.

The behavior of fluid interaction with these surfaces was studied by attaching CNT arrays (and subjecting them to identical surface treatments) on porous RVC foam samples that could be inserted in a cylindrical water flow cartridge. Pressure drop per unit length (ΔP/L) across these samples were measured as a function of water flow velocity (4 Assuming the fluid flow through porous materials is incompressible, ΔP vs. ϑ graphs were obtained for all the concerned porous structures. In this experiment, water flow rate was controlled by a master flux L/S (model 7518-10) controller. The filtration system included a porous structure fitted inside acrylic casing. Fluid in the storage container (water in this experiment) was fed into the porous material using a pump drive. The storage container, pump drive, filtration system, and collecting tank all were connected in series via ⅛ inch latex tubing. A manometer was connected at the fluid entry side while leaving the exit end open to the atmosphere. The height difference (Δh) in manometer “U” column was measured and the pressure drop (ΔP=ρ×g× Δh) was calculated, where ρ is the fluid density, and g is the gravitational constant. Superficial fluid velocity (ϑ) or volumetric flux (q) is (vol. flow rate (Q)/cross sectional area (A)), and was taken on the X-axis. Fluid volume flow rate was varied for specified intervals and pressure drop per length (ΔP/L) was measured at each flow rate.

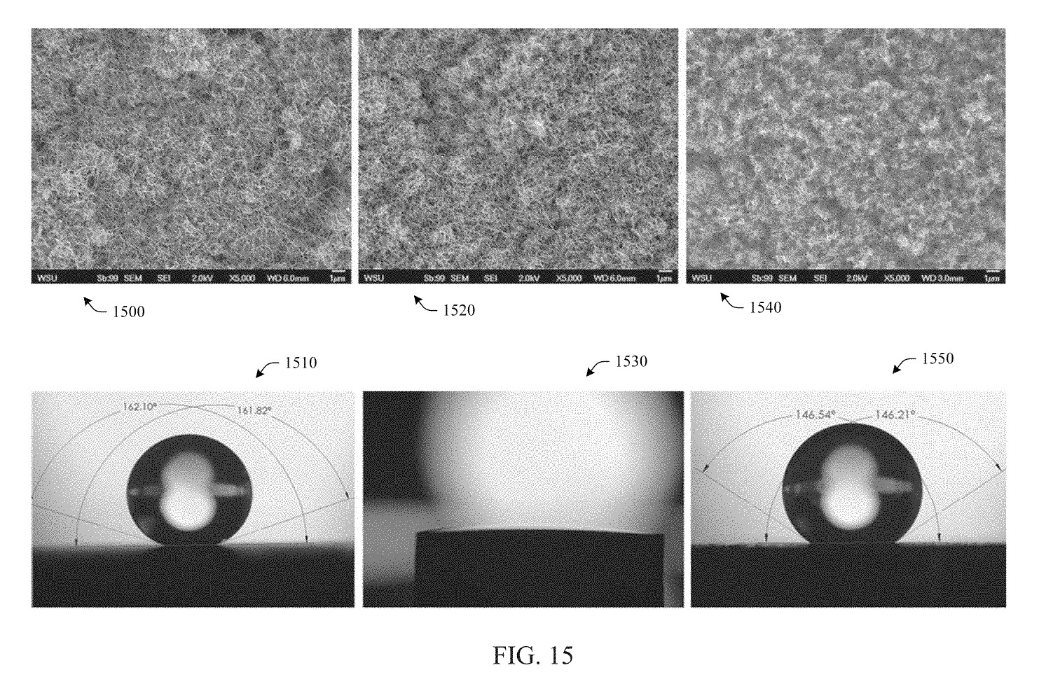

In the plasma oxygen experiment, carbon nanotube arrays grafted on HOPG substrate (CNT-HOPG) were used as the base-line surface and fabricated as explained above, and surface wettability was quantified as contact angle (CA) and measured as described earlier. Wettability of any surface is governed by both morphological structure and chemistry. The CNT-HOPG surface was treated with 115 W oxygen plasma for 10 sec as explained above. Care was taken, since dry etching with oxygen and ozone plasmas have been reported to cause structural damage to CNT due to defect creation and rupture of the hexagonal network. In connection with these experiments, this effect was observed at higher exposure times and microwave powers, but not at the operating microwave power and exposure time discussed herein. FIG. 15 shows microstructures of CNT-HOPG at 1500 and oxygen treated CNT-HOPG (O-CNT-HOPG) surface at 1510, and it can be seen that there was no noticeable change in morphology. It can be seen that CA was drastically decreased, from 162.5±2° before treatment, as seen at 1520, to less than 10° with 10 sec oxygen treatment, as seen at 1530. When CA is too low, water easily seeps into the CNT carpet, and quantitative accuracy is questionable, hence only the largest measurable water drop is shown at 1530. The conversion from super-hydrophobic to hydrophilic surface is clear.

Air annealing of O-CNT-HOPG sample was performed as explained above, and this surface (air-O-CNT-HOPG) showed no visible microstructural changes, as seen at 1540. However, hydrophobicity of CNT grafted surface was restored as evident from CA measurement. The measured CA on air-O-CNT-HOPG surface was 150±10°, as seen in 1550. Earlier studies that have treated loose CNT with oxygen and ozone plasmas have shown reversal of wettability after high temperature vacuum annealing or hydrogen treatments. These experiments have been able to create hydrophilic surface with very gentle room temperature microwave plasma that can be reversed at relatively mild temperature in air, making the process significantly more scalable. In fact, this hydrophilic-hydrophobic treatment could be repeated on the same sample multiple times, indicating that it does not compromise the sample structure in any way.

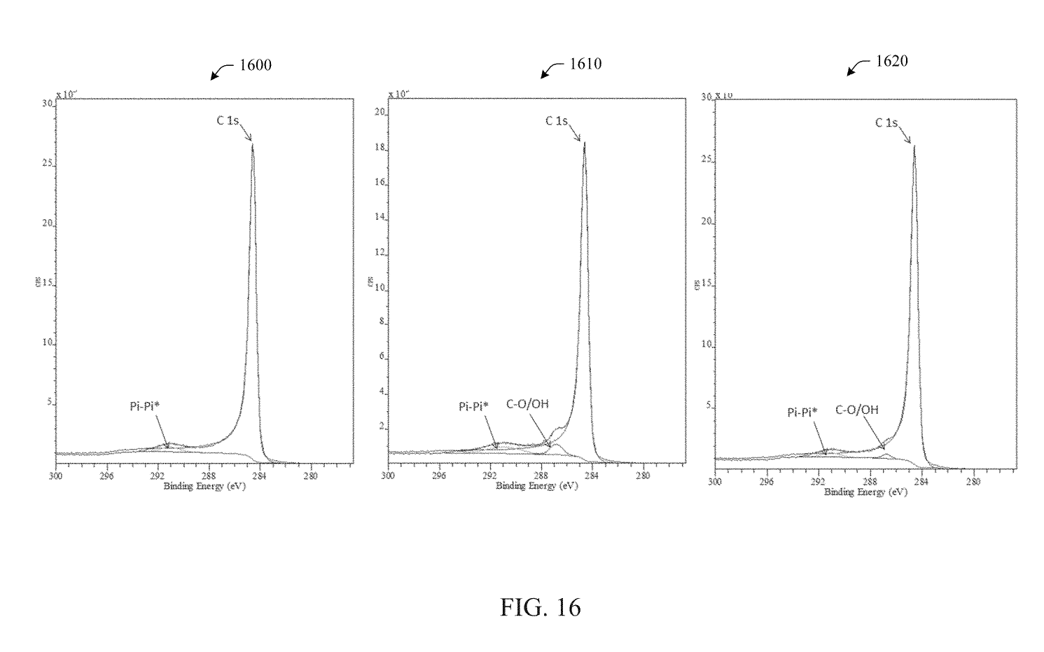

Influence of surface chemistry on wettability of surface modified CNT-HOPG was studied using XPS. FIG. 16 illustrates the peak analysis of CNT-HOPG, O-CNT-HOPG, and air annealed-O-CNT-HOPG, at 1600, 1610, and 1620, respectively. The deconvoluted peak positions and their corresponding concentrations, averaged over two regions, are shown in Table 1, below, showing the influence of the C/O ratio on contact angle and component based quantification. It is clear that oxygenated carbon component was introduced with plasma oxygen treatment, and was significantly reduced with air annealing. Conversely, the corresponding CA was decreased to less than 10° with oxygen etching and increased by to 146° with annealing. From this analysis, it can be seen that the oxygenated carbon species were closely related to wettability while physisorbed oxygen (included in the total O1s peak) is not a reliable indicator. In the experiments, it was seen that hydrophilic behavior created by microwave oxygen plasma can easily be reverted to hydrophobicity with simple heating, unlike earlier studies with RF plasma and Oxygen ozone etched processes, which seem to require high temperature heating in vacuum or hydrogen. The easy transition between super hydrophobic to super hydrophilic can have important applications, such as self-cleaning surface, hydro dynamic skin friction reduction, drug delivery and fluid separation devices. On the flip side, if applications require sustained hydrophilic behavior even at higher temperatures, this approach may not be a suitable one. Such situations will need a technique for more permanent surface modification, as discussed below.

TABLE 1
Influence of C 1s photoelectron peak components on contact angle

| | | | | Air |
|---|---|---|---|---|
| | | | Oxygen- | annealed- |
| | | CNT-HOPG | CNT-HOPG | Oxygen- |
| | BE positions | (%), | (%) | CNT-HOPG |
| | (eV) | θ = 164 ± 2° | θ = ≤10° | θ = 150 ± 2° |
| C asymmetric | 284.6 | 97.7 | 92.1 | 97.2 |
| peak | | | | |
| C—O/OH | 286.6 ± 0.1 | below | 3.0 | 0.9 |
| (hydroxyl) | | detection | | |
| π → π* shake | 291 ± 0.1 | 2.3 | 4.9 | 1.9 |
| up peaks | | | | |

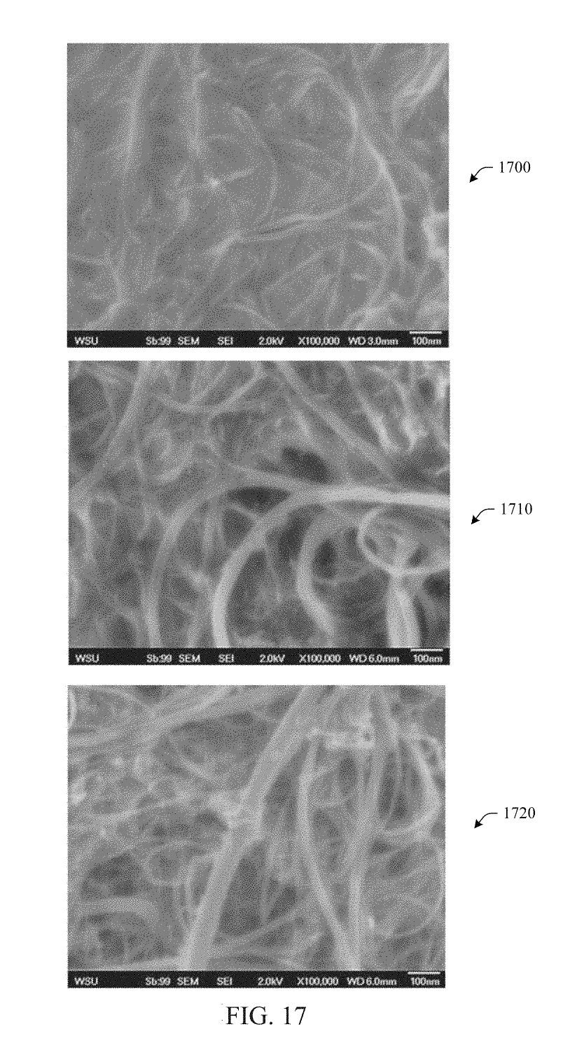

In a polar oxide coating experiment, CNT-HOPG surfaces were dip-coated with varying ratios of sol-gel solution as explained above. FIG. 17 illustrates the microstructure of silica coated CNT-HOPG surfaces (silica-CNT-HOPG). It can be seen that a concentrated silica solution resulted in silica clumping, more like a silica-CNT composite microstructure, as seen at 1700. However, as the concentration of TEOS was decreased from 1700 (TEOS to water ratio of 1:8) to 1710 (ratio of 1:33) and to 1720 (ratio of 1:50), silica thickness was controllable to the thickness scales suitable for CNT surfaces. At the concentration ratio of 1:50, individual CNT morphology was retained with a thin layer of coating, as seen in 1720. This was selected as a final recipe for the remainder of the experiment. It is to be understood that although this specific ratio was used in the experiments, in various aspects, different ratios can be used, for example, between about 1:40 and about 1:60, between about 1:30 and about 1:70, etc. Additionally, these ratios may depend on other characteristics, such as materials selected for nanotubes, coatings, etc. For example, simpler surface with sharper and more sparse nanotubes may benefit from higher ratios than 1:50, such as around 1:30 or higher, where more complex surfaces with longer and denser nanotubes may benefit from lower ratios, such as around 1:70 or lower, and longer process times.

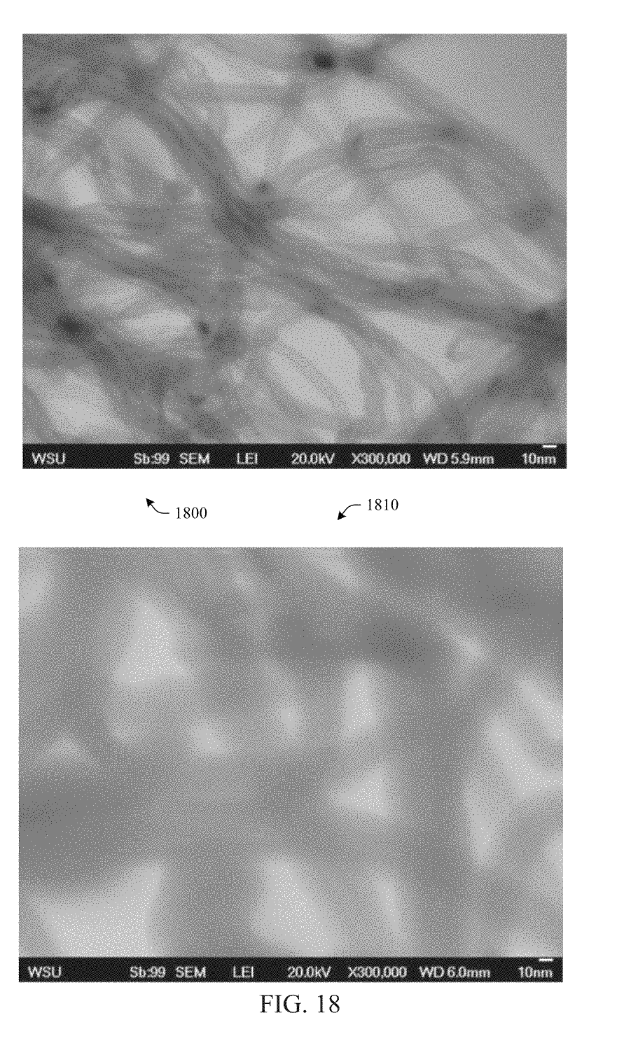

Transmission mode microstructures were taken using a STEM detector. For STEM analysis, a layer of silica coated CNT/graphite was peeled off and loaded on copper grids. FIG. 18 illustrates the STEM images of both as-grown CNT and silica-CNT in 1800 and 1810, respectively. In 1800, the thin walls of CNT and hollow space can be clearly seen. The average size of CNT outer and inner diameter was 15 nm and 10 nm respectively. In the case of silica coated CNT, a fuzzy and texture-less layer was seen on the CNT. Silica grown by sol-gel is expected to be amorphous without any long range order. The thickness of this region varied greatly and few thickly coated regions were observed. In various aspects not pursued in the experiments, one or more surfactants can be added to the sol-gel precursor in order to obtain more uniform silica layers. However, it can be seen from these results that the CNTs were coated with an additional film.

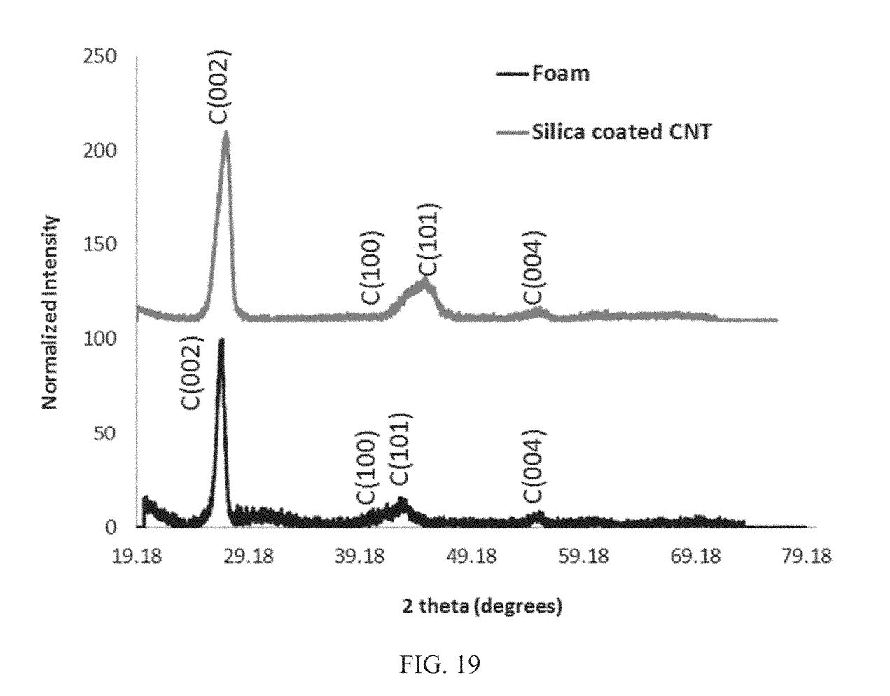

Further analysis was done to study the crystal structure of silica coated CNT-carbon surface. From the XRD patterns shown in FIG. 19 it can be seen that the diffraction peaks for both as grown CNT and silica-CNT were exactly identical and related to graphite and CNT only. The absence of any diffraction peaks from the presence of silica indicated that the formed silica was 100% amorphous. This result was in agreement with previous studies related to silica films prepared on carbon/graphite via this method.

For surface chemistry analysis of the silica film, detailed XPS analyses was performed on baseline CNT-HOPG and silica-CNT-HOPG. From this analysis, it was observed that the silica coated surfaces were free of reaction byproducts and contaminants. The region based semi quantification studies are shown in Table 2, below.

TABLE 2
Region based semi quantification CNT-HOPG and silica-CNT-HOPG

| | | |
|---|---|---|
| BE (eV) | FWHM | At % |

CNT-HOPG

| | | | |
|---|---|---|---|
| C 1s | 284.6 | 0.655 | 98.97 |
| O 1s | 533.2 | 2.048 | 1.03 |

Silica-CNT-HOPG

| | | | |
|---|---|---|---|
| C 1s | 284.6 | 0.643 | 68.75 |
| O 1s | 533 | 1.71 | 21.07 |
| Si 2p | 103.9 | 1.676 | 10.18 |

Upon silica coating, there was no observable difference either in binding energy (BE) and shape of C is peak is before and after silica coating.

From detailed analysis of peak positions and shapes, and comparing them with peaks from standard silica films as well as quartz glass, it was concluded that formed silica was chemically identical to pure silicon dioxide (Si⁴⁺/SiO₂).

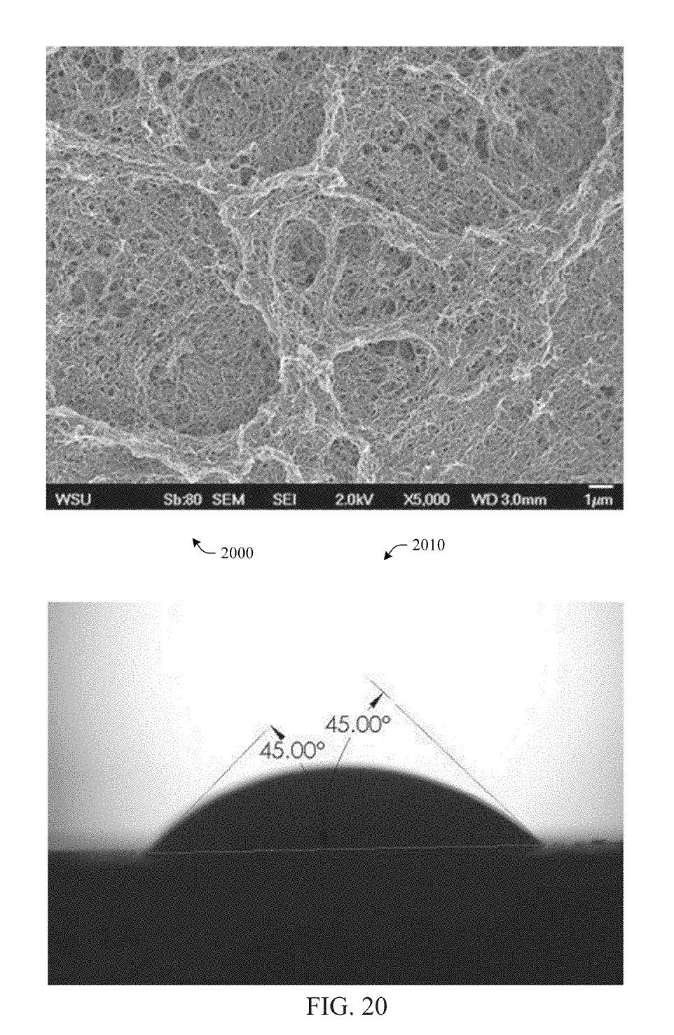

FIG. 20 illustrates the microstructure 2000 and contact angle 2010 of a silica-CNT-HOPG sample from the experiments. Water CA was measured after silica coating on CNT-HOPG and observed to be 45±5.5°, seen in 2010 which was a significant reduction from CA on CNT-HOPG (160°). Strong positive correlation between silica concentration and wettability was also observed, similar to observations made on conventional sol-gel thin films of silica. Unlike plasma treatments, silica coatings induce permanent wettability. However, any liquid based coating on tall CNT arrays will tend to change its global structure due to collapse of the carpet during water evaporation, as seen in FIG. 2000. However, it does not change the CNT stands or any functionality at the local scale. These silica coated CNT-carbon surfaces enable more efficient functionalization with nanoparticles, as well as and better utilization of available surface area for aqueous reactions.

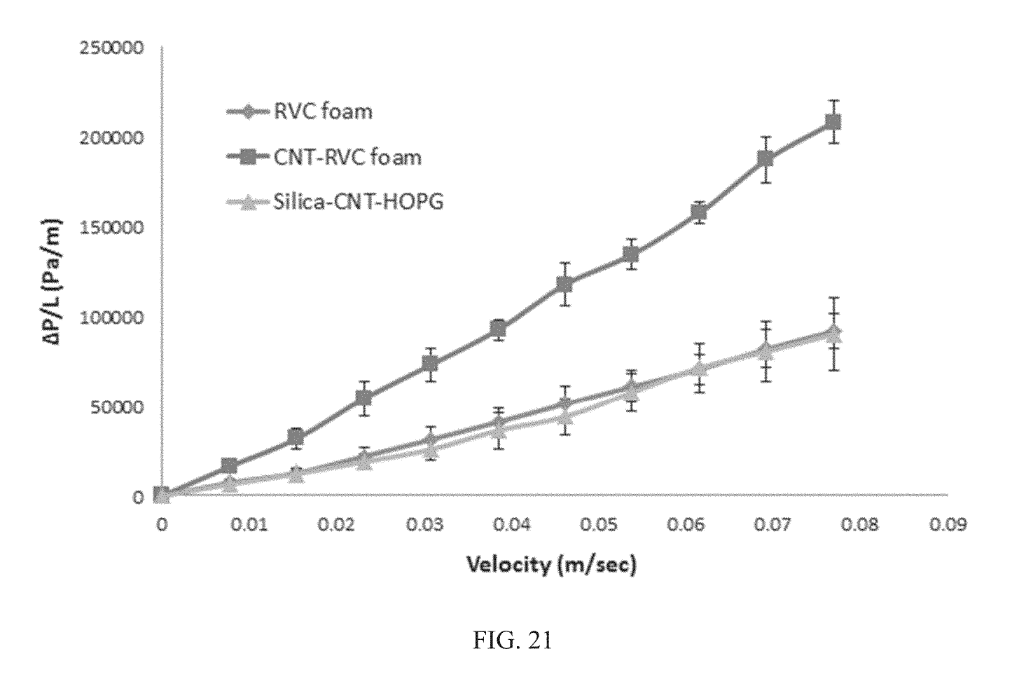

To investigate the influence of wettability on fluid flow characteristics, reticulated carbon foam (RVC foam) was used as a porous substrate suitable for many membrane or filtration related applications. Fluid flow characteristic graphs (Δp/L Vs υ) were measured for three different samples: (1) bare RVC foam, (2) CNT grafted RVC foam (CNT-RVC), and (3) silica coated CNT-RVC (silica-CNT-RVC). FIG. 21 illustrates the average ΔP/L for a given volumetric flux over three samples for each category. The ΔP/L plot indicates the energy required to maintain the given flow rate. The governing equation is:

$$\frac{Δ P}{L} = \frac{α {{S}_{v - solid}^{2} ( 1 - ɛ )}^{2}}{{ɛ}^{3}} μϑ + \frac{β {S}_{v - solid} ( 1 - ɛ )}{{ɛ}^{3}} {ρϑ}^{2} + \frac{{P}_{c}^{e}}{L} \qquad ( 1 )$$

The terms S_{v-solid} and ε are specific surface area and fractional open porosity respectively. α and β are fit coefficients for viscous and inertial terms respectively. (P_{c}^{e}/L) is the “effective capillary pressure” term, which will be negative for hydrophilic surfaces and positive for hydrophobic surface. Some of the governing parameters that can directly influence the ΔP/L are geometrical features of the porous media such as porosity (ε), specific surface area at micro-scale level (S), and effective capillary pressure, which is qualitatively related to surface wettability (θ) in this case. For a planar cylindrical surface, this would be directly proportional to cos θ, but more complicated modeling would be required for this type of hierarchical surface. In this experiment, the varying parameters between various structures were ε, S, and θ.

From the figure it can be seen that, at any given υ, ΔP/L increases with CNT grafting and decreases with silica coating or improved wettability. While the experiments did not develop a quantitative estimate in these complex multiscale solids, the general rule is that lower c (or lower S) results in higher pressure drops. Also, flow through micro channels is dominated by capillaries and fluid slips. It can be clearly seen that more energy is required for the fluid navigate through CNT-RVC foam compared to untreated foam. This is due to reduced porosity caused by CNT grafting, and further reduction in fluid channel due to super hydrophobicity of the surface. However, when these surfaces were coated with silica, apparent fluid flow channel seems to be increased, aided by capillary driven flows leading them to have lower ΔP/L. This experiment shows that the effective surface area increases with CNT attachment and fluid flow through the CNT carpets can be increased with hydrophilic coating. This can have important implications in applications related to flow of water or biological fluids across such surfaces.

In accordance with aspects of the subject innovation, in wettability experiments, two kinds of surface modification techniques were tested on CNT grafted carbon surfaces: (1) plasma oxygen etching via microwave plasma, and (2) silica coatings via liquid based sol gel method. The oxygen plasma technique improved the surface wettability “temporarily” due to the absorption of hydroxyl groups (C—O/OH). However, hydrophobicity of the surface could be restored due to desorption of C—O/OH groups with application of heat. While switchable wettability on CNT grafted surfaces can have uses in self-cleaning, liquid separation devices, applications requiring permanent wettability can employ alternative techniques. Sol-gel based silica coatings are more suitable for that. Microstructure and crystallographic studies showed the silica coatings to be amorphous. Surface chemistry studies showed that the coatings are clean with no trace elements and equivalent to pure silicon dioxides with BE of Si 2p (103.9 eV) and O is (533 eV). Chemistry of coating process was also studied at different stages of coating process and observed that electronic states of Si and O did not change throughout the process, which enables lower annealing temperatures in future. Water CA results showed that wettability of silica coated CNT-carbon was permanently hydrophilic. Fluid flow tests showed that effective surface area is increased due to the improved wettability. These results have applications in liquid purification devices, NPs functionalization, and structural composites.

What has been described above includes examples of the innovation. It is, of course, not possible to describe every conceivable combination of components or methodologies for purposes of describing the subject innovation, but one of ordinary skill in the art may recognize that many further combinations and permutations of the innovation are possible. Accordingly, the innovation is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A multilayer hierarchical structure for promoting cell differentiation comprising:
at least one scaffold comprising;
a scaffold surface;
a modified layer comprising at least one plurality of nanoscale attachments strongly bonded to the modified layer, wherein the nanoscale attachments form a nanostructured nanotube carpet; and
microscale features selected from interconnected pores, highly aligned fibers, or a combination thereof, interfaced with the at least one plurality of nanoscale attachments.

2. The structure of claim 1 wherein the nanoscale attachment is a carbon nanotube, a Si-carbon nanotube, or a combination thereof.

3. The structure of claim 1 wherein the scaffold is selected from a porous carbon foam, an aligned carbon fiber mat, or a combination thereof.

4. The structure of claim 3 wherein the scaffold is composed of repeating small structural units to create a three-dimensional architecture.

5. The structure of claim 4, wherein the repeating small structural units are comprised of interconnected porous carbon foams or aligned carbon fiber mats,
wherein carbon foams have 200-500 μm size micro-pores with wall width of 50-80 μm as structural repeating units, and
wherein the aligned carbon fiber mats have carbon microfibers with diameters of about 5-6 μm as structural repeating units.

6. The structure of claim 1, wherein the nanoscale attachments are multi-walled carbon nanotubes.

7. The structure of claim 1 wherein the modified layer comprises surface functionalization.

8. The structure of claim 7 wherein the nanoscale attachments are functionalized nanoscale attachments.

9. The structure of claim 8 wherein the nanoscale attachments are functionalized with amorphous silica (SiOx).

10. The structure of claim 8, wherein the wherein the functional species are functionalized with palladium nanoparticles.

11. The structure of claim 8 wherein the nanoscale attachments promote cell differentiation of myoblasts.

12. The structure of claim 8 wherein the nanoscale attachments promote cell differentiation of neuronal cells.

13. The structure of claim 8 wherein the nanoscale attachments promote cell differentiation of cardiac cells.

14. A method of forming a multiscale hierarchical scaffold comprising:
selecting and preparing a parent substrate;
modifying the substrate surface to form an intermediate layer;
attaching at least one plurality of nanoscale attachments onto the intermediate layer to form a nanostructured carbon nanotube carpet; wherein the steps of forming the intermediate layer and attaching the nanoscale attachments employs one or more of (a) wet chemistry, (b) chemical vapor deposition, (c) plasma deposition, (d) atomic layer deposition, (e) physical vapor deposition, (f) controlled environment heating, or a combination thereof; and
interfacing a microscale aligned fibrous architecture with the nanostructured carbon nanotube carpet.

15. The method of claim 14, further comprising coating the nanoscale attachments with a functional species

16. The method of claim 15, wherein coating the nanoscale attachments comprises heating the hierarchical structure in a microwave oxygen plasma.

17. The method of claim 15, wherein coating the nanoscale attachments comprises:
preparing an acid catalyzed oxide gel;
dip coating the hierarchical structure in the oxide gel;
drying the hierarchical substrate; and
annealing the hierarchical substrate.

18. The method of claim 14, wherein the parent substrate is further processed into two types of multiscale hierarchical scaffolds; (i) a porous carbon foam and (ii) an aligned carbon fiber mat.

19. The method of claim 14, wherein the nanostructured carbon nanotube carpet is grown on a micro-pore wall of a porous foam or an aligned fiber mat.

20. The method of claim 19, wherein the foam is a carbon foam and the fiber mat is a carbon fiber mat.
